(19) United States
(12) Patent Application Publication
(10) Pub. No.: US 2005/0032794 A1
(43) Pub. Date: Feb. 10, 2005

(54) Diamine derivatives of quinone and uses thereof

(76) Inventors: Janak K. Padia, Germantown, MD (US); Sean O'Brien, Gaithersburg, MD (US); Jiemin Lu, Germantown, MD (US); Stanislaw Pikul, Germantown, MD (US)

Correspondence Address: Alan J. Grant, Esq.;c/o Carella, Byrne, Bain, Gilfillan,, Cecchi, Stewart & Olstein, 6 Becker Farm Road, Roseland, NJ, 07068, US

(21) Appl. No.: 10/758,521

(22) Filed: Jan. 15, 2004

Related U.S. Application Data
(60) Provisional application No. 60/492,521, filed on Aug. 5, 2003.
(60) Provisional application No. 60/523,477, filed on Nov. 19, 2003.

(51) Int. Cl. C07D 265/36; C07D 215/12; A61K 31/538; A61K 31/517; A61K 31/498; A61K 31/47; A61K 31/165

(52) U.S. Cl. 514/230.5; 514/266.1; 514/248; 514/249; 514/311; 514/310; 514/619; 544/105; 544/235; 544/283; 544/353; 546/139; 546/176; 552/292

(57) ABSTRACT

Diamine derivatives of quinones, and related compounds, including salts thereof, that modulate the levels of gene expression in cellular systems, such as cancer cells, are disclosed, along with methods for preparing such compounds and derivatives, as well as pharmaceutical compositions containing these compounds and derivatives as active ingredients. Methods of using these as compounds and derivatives as therapeutic agents are also described.

This application claims priority of U.S. Provisional Application Ser. No. 60/492,521, filed 5 Aug. 2003, and 60/523,477, filed 19 Nov. 2003, the disclosures of which are hereby incorporated by reference in their entirety.

### FIELD OF THE INVENTION

The present invention relates to chemical agents affecting levels of gene expression in cellular systems, including cancer cells. In particular, the present invention relates to derivatives of quinone moiety, processes for their preparation, their use as antitumor drugs and pharmaceutical compositions containing them as active ingredients.

### BACKGROUND OF THE INVENTION

Screening assays for novel drugs are based on the response of model cell based systems in vitro to treatment with specific compounds. Various measures of cellular response have been utilized, including the release of cytokines, alterations in cell surface markers, activation of specific enzymes, as well as alterations in ion flux and/or pH. Some such screens rely on specific genes, such as oncogenes or tumor suppressors.

Our approach to screening small molecule compounds as potential anticancer drugs is based on the idea that for each specific tumor type, a unique signature set of genes, that are differentially expressed in tumor cells if compared to corresponding normal cells, can be established. The relatively small signature set, containing 10-30 genes, allows for easy, high throughput screening for compounds that can reverse the gene expression profile from patterns typical for cancer cells to patterns seen in normal cells. As a part of our efforts to provide new diversified compounds for high throughput gene expression screening, we designed and synthesized a number of novel derivatives of quinones. Gene expression screening and subsequent cytotoxicity screening revealed that some of the compounds possess biological activity. Consequent, more detailed structure-activity relationship studies led to the discovery of compounds of formula I as new small molecule agents having antineoplastic activity.

### BRIEF SUMMARY OF THE INVENTION

In one aspect, the present invention relates to novel organic compounds, derivatives of quinone, that have the ability to function as gene expression modulators for genes found in cancer cells, especially genes involved in misregulated signal transduction pathways typical for cancer such as colon and breast cancers.

In one embodiment of the present invention, the compounds disclosed herein are able to up regulate genes found to be up regulated in normal (i.e., non-cancerous) cells versus cancer cells, especially colon and breast cancer cells, thereby producing an expression profile for said gene(s) that more resembles the expression profile found in normal cells. In another embodiment, the compounds disclosed herein are found to down regulate genes found to be up regulated in cancer cells, especially colon and breast cancer cells, relative to normal (i.e., non-cancerous) cells thereby producing an expression profile for said gene(s) that more resembles the expression profile found in normal cells. Thus, in addition to activity in modulating a particular gene that may or may not have a major role in inducing or sustaining a cancerous condition, the agents disclosed herein also find value in regulating a set of gene whose combined activity is related to a disease condition, such as cancer, especially colon and breast cancer, including adenocarcinoma of the colon. Thus, while an overall set of genes is modulated, the effect of modulating any subset of these may be disproportionately large or small with respect to the effect in ameliorating the overall disease process. Consequently, different disease conditions may rely on different subsets of genes to be active or inactive as a basis for the overall disease process.

Thus, the present invention relates to novel organic compounds that have the ability to function as gene modulators for genes found in normal (i.e., non-cancer) cells and which genes are found to be up regulated or down regulated in normal cells, especially colon and breast cells. Such an effect may prevent a disease condition, such as cancer, from arising in those otherwise more susceptible to such a condition. In one such embodiment, administration of one or more of the agents disclosed herein may succeed in preventing a cancerous condition from arising.

In other embodiments, the agents disclosed herein find use in combination with each other as well as with other agents, such as where a mixture of one or more of the agents of the present invention are given in combination or where one or more of the agents disclosed herein is given together with some other already known therapeutic agent, possibly as a means of potentiating the affects of such known therapeutic agent or vice versa.

The present invention also relates to processes of preventing or treating disease conditions, especially cancer, most especially colon and breast cancer, by administering to a subject, such as a mammal, especially a human, a therapeutically active amount of one or more of the agents disclosed herein, including where such agents are given in combination with one or more known therapeutic agents.

### DEFINITIONS

The following is a list of definitions for terms used herein.

“Acyl” or “carbonyl” is a radical formed by removal of the hydroxy from a carboxylic acid (i.e., R—C(═O)—). Preferred acyl groups include (for example) acetyl, formyl, and propionyl.

“Alkyl” is a saturated hydrocarbon chain having 1 to 15 carbon atoms, preferably 1 to 10, more preferably 1 to 4 carbon atoms. “Alkene” is a hydrocarbon chain having at least one (preferably only one) carbon-carbon double bond and having 2 to 15 carbon atoms, preferably 2 to 10, more preferably 2 to 4 carbon atoms. “Alkyne” is a hydrocarbon chain having at least one (preferably only one) carbon-carbon triple bond and having 2 to 15 carbon atoms, preferably 2 to 10, more preferably 2 to 4 carbon atoms. Alkyl, alkene and alkyne chains (referred to collectively as “hydrocarbon chains”) may be straight or branched and may be unsubstituted or substituted. Preferred branched alkyl, alkene and alkyne chains have one or two branches, preferably one branch. Preferred chains are alkyl. Alkyl, alkene and alkyne hydrocarbon chains each may be unsubstituted or substituted with from 1 to 4 substituents; when substituted, preferred chains are mono-, di-, or tri-substituted. Alkyl, alkene and alkyne hydrocarbon chains each may be substituted with halo, hydroxy, aryloxy (e.g., phenoxy), heteroaryloxy, acyloxy (e.g., acetoxy), carboxy, aryl (e.g., phenyl), heteroaryl, cycloalkyl, heterocycloalkyl, spirocycle, amino, amido, acylamino, keto, thioketo, cyano, or any combination thereof. Preferred hydrocarbon groups include methyl, ethyl, propyl, isopropyl, butyl, vinyl, allyl, butenyl, and exomethylenyl.

Also, as referred to herein, a “lower” alkyl, alkene or alkyne moiety (e.g., “lower alkyl”) is a chain comprised of 1 to 6, preferably from 1 to 4, carbon atoms in the case of alkyl and 2 to 6, preferably 2 to 4, carbon atoms in the case of alkene and alkyne.

“Alkoxy” is an oxygen radical having a hydrocarbon chain substituent, where the hydrocarbon chain is an alkyl or alkenyl (i.e., —O-alkyl or -—O-alkenyl). Preferred alkoxy groups include (for example) methoxy, ethoxy, propoxy and allyloxy. p “Aryl” is an aromatic hydrocarbon ring. Aryl rings are monocyclic or fused bicyclic ring systems. Monocyclic aryl rings contain 6 carbon atoms in the ring. Monocyclic aryl rings are also referred to as phenyl rings. Bicyclic aryl rings contain from 8 to 17 carbon atoms, preferably 9 to 12 carbon atoms, in the ring. Bicyclic aryl rings include ring systems wherein, one ring is aryl and the other ring is aryl, cycloalkyl, or heterocycloakyl. Preferred bicyclic aryl rings comprise 5-, 6- or 7-membered rings fused to 5-, 6-, or 7-membered rings. Aryl rings may be unsubstituted or substituted with from 1 to 4 substituents on the ring. Aryl may be substituted with halo, cyano, nitro, hydroxy, carboxy, amino, acylamino, alkyl, heteroalkyl, haloalkyl, phenyl, aryloxy, alkoxy, heteroalkyloxy, carbamyl, haloalkyl, methylenedioxy, heteroaryloxy, or any combination thereof. Preferred aryl rings include naphthyl, tolyl, xylyl, and phenyl. The most preferred aryl ring radical is phenyl.

“Aryloxy” is an oxygen radical having an aryl substituent (i.e., —O-aryl). Preferred aryloxy groups include (for example) phenoxy, napthyloxy, methoxyphenoxy, and methylenedioxyphenoxy.

“Cycloalkyl” is a saturated or unsaturated hydrocarbon ring. Cycloalkyl rings are not aromatic. Cycloalkyl rings are monocyclic, or are fused, spiro, or bridged bicyclic ring systems. Monocyclic cycloalkyl rings contain from about 3 to about 9 carbon atoms, preferably from 3 to 7 carbon atoms, in the ring. Bicyclic cycloalkyl rings contain from 7 to 17 carbon atoms, preferably from 7 to 12 carbon atoms, in the ring. Preferred bicyclic cycloalkyl rings comprise 4-, 5- 6- or 7-membered rings fused to 5-, 6-, or 7-membered rings. Cycloalkyl rings may be unsubstituted or substituted with from 1 to 4 substituents on the ring. Cycloalkyl may be substituted with halo, cyano, alkyl, heteroalkyl, haloalkyl, phenyl, keto, hydroxy, carboxy, amino, acylamino, aryloxy, heteroaryloxy, or any combination thereof. Preferred cycloalkyl rings include cyclopropyl, cyclopentyl, and cyclohexyl.

“Halo” or “halogen” is fluoro, chloro, bromo or iodo. Preferred halo are fluoro, chloro and bromo; more preferred typically are chloro and fluoro, especially fluoro.

“Haloalkyl” is a straight, branched, or cyclic hydrocarbon substituted with one or more halo substituents. Preferred are C₁-C₁₂ haloalkyls; more preferred are C₁-C₆ haloalkyls; still more preferred still are C₁-C₃ haloalkyls. Preferred halo substituents are fluoro and chloro. The most preferred haloalkyl is trifluoromethyl.

“Heteroatom” is a nitrogen, sulfur, or oxygen atom. Groups containing more than one heteroatom may contain different heteroatoms.

“Heteroalkyl” is a saturated or unsaturated chain containing carbon and at least one heteroatom, wherein no two heteroatoms are adjacent. Heteroalkyl chains contain from 2 to 15 member atoms (carbon and heteroatoms) in the chain, preferably 2 to 10, more preferably 2 to 5. For example, alkoxy (i.e., —O-alkyl or —O-heteroalkyl) radicals are included in heteroalkyl. Heteroalkyl chains may be straight or branched. Preferred branched heteroalkyl have one or two branches, preferably one branch. Preferred heteroalkyl are saturated. Unsaturated heteroalkyl have one or more carbon-carbon double bonds and/or one or more carbon-carbon triple bonds. Preferred unsaturated heteroalkyls have one or two double bonds or one triple bond, more preferably one double bond. Heteroalkyl chains may be unsubstituted or substituted with from 1 to 4 substituents. Preferred substituted heteroalkyl are mono-, di-, or tri-substituted. Heteroalkyl may be substituted with lower alkyl, haloalkyl, halo, hydroxy, aryloxy, heteroaryloxy, acyloxy, carboxy, monocyclic aryl, heteroaryl, cycloalkyl, heterocycloalkyl, spirocycle, amino, acylamino, amido, keto, thioketo, cyano, or any combination thereof. Where a group is described, for example, as an alkyl derivative, such as “-ethylpyridine” the dash “-” indicate point of attachment of the substituent. Thus, “-ethylpyridine” means attachment of ethylpyridine via the ethyl portion of the group whereas “ethylpyridine-” means attachment via the pyridinyl ring.

“Heteroaryl” is an aromatic ring containing carbon atoms and from 1 to about 6 heteroatoms in the ring. Heteroaryl rings are monocyclic or fused bicyclic ring systems. Monocyclic heteroaryl rings contain from about 5 to about 9 member atoms (carbon and heteroatoms), preferably 5 or 6 member atoms, in the ring. Bicyclic heteroaryl rings contain from 8 to 17 member atoms, preferably 8 to 12 member atoms, in the ring. Bicyclic heteroaryl rings include ring systems wherein one ring is, heteroaryl and the other ring is aryl, heteroaryl, cycloalkyl, or heterocycloalkyl. Preferred bicyclic heteroaryl ring systems comprise 5-, 6- or 7-membered rings fused to 5-, 6-, or 7-membered rings. Heteroaryl rings may be unsubstituted or substituted with from 1 to 4 substituents on the ring. Heteroaryl may be substituted with halo, cyano, nitro, hydroxy, carboxy, amino, acylamino, alkyl, heteroalkyl, haloalkyl, phenyl, alkoxy, aryloxy, heteroaryloxy, or any combination thereof. Preferred heteroaryl rings include, but are not limited to, the following:

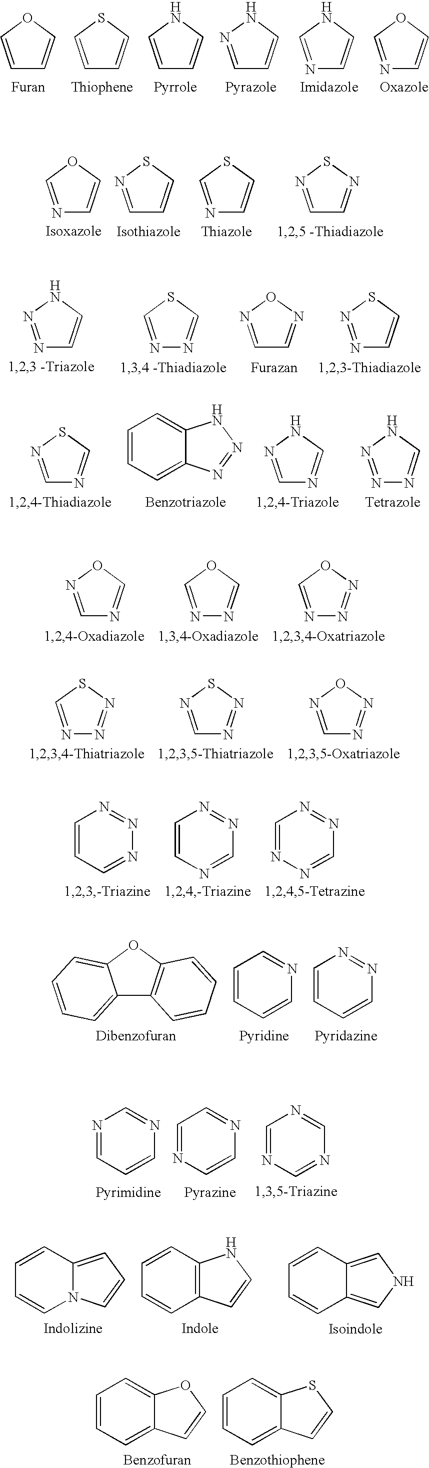

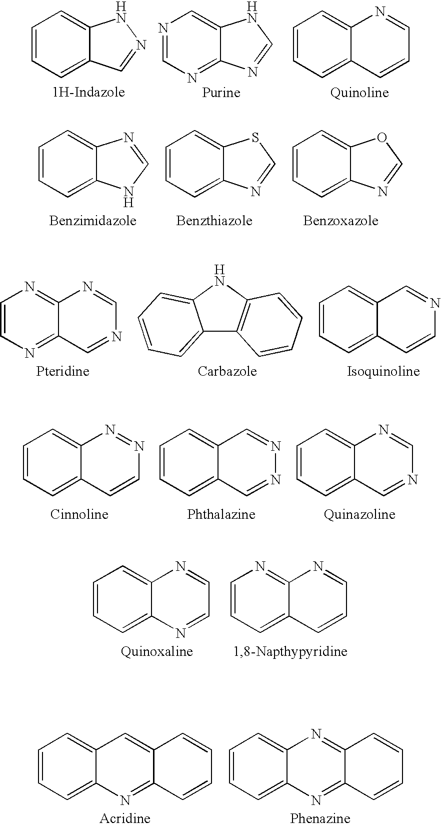

“Heteroaryloxy” is an oxygen radical having a heteroaryl substituent (i.e., —O-heteroaryl). Preferred heteroaryloxy groups include (for example) pyridyloxy, furanyloxy, (thiophene)oxy, (oxazole)oxy, (thiazole)oxy, (isoxazole)oxy, pyrmidinyloxy, pyrazinyloxy, and benzothiazolyloxy.

“Heterocycloalkyl” is a saturated or unsaturated ring containing carbon atoms and from 1 to about 4 (preferably 1 to 3) heteroatoms in the ring. Heterocycloalkyl rings are not aromatic. Heterocycloalkyl rings are monocyclic, or are fused, bridged, or spiro bicyclic ring systems. Monocyclic heterocycloalkyl rings contain from about 3 to about 9 member atoms (carbon and heteroatoms), preferably from 5 to 7 member atoms, in the ring. Bicyclic heterocycloalkyl rings contain from 7 to 17 member atoms, preferably 7 to 12 member atoms, in the ring. Bicyclic heterocycloalkyl rings contain from about 7 to about 17 ring atoms, preferably from 7 to 12 ring atoms. Bicyclic heterocycloalkyl rings may be fused, spiro, or bridged ring systems. Preferred bicyclic heterocycloalkyl rings comprise 5-, 6- or 7-membered rings fused to 5-, 6-, or 7-membered rings. Heterocycloalkyl rings may, be unsubstituted or substituted with from 1 to 4 substituents on the ring. Heterocycloalkyl may be substituted with halo, cyano, hydroxy, carboxy, keto, thioketo, amino, acylamino, acyl, amido, alkyl, heteroalkyl, haloalkyl, phenyl, alkoxy, aryloxy or any combination thereof. Preferred substituents on heterocycloalkyl include halo and haloalkyl. Preferred heterocycloalkyl rings include, but are not limited to, the following:

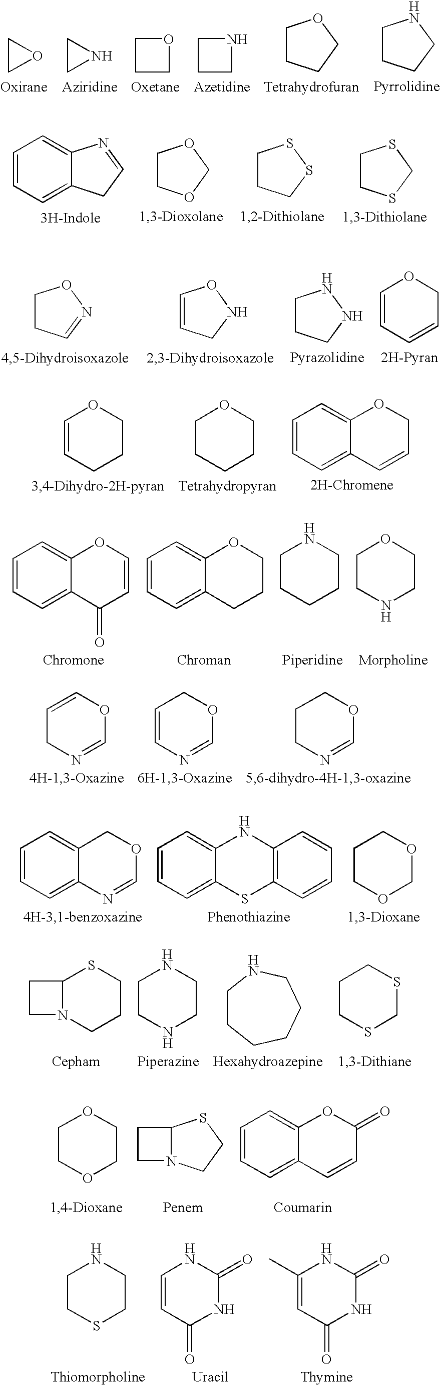

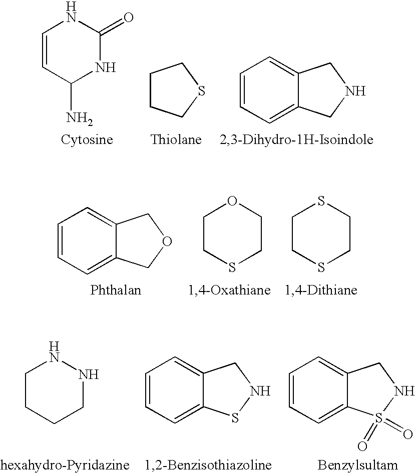

While alkyl heteroalkyl, cycloalkyl, and heterocycloalkyl groups may be substituted with hydroxy, amino, and amido groups as stated above, the following are not envisioned in the invention:

Enols (OH attached to a carbon bearing a double bond).

Amino groups attached to a carbon bearing a double bond (except for vinylogous amides).

More than one hydroxy, amino, or amido attached to a single carbon (except where two nitrogen atoms are attached to a single carbon atom and all three atoms are member atoms within a heterocycloalkyl ring).

Hydroxy, amino, or amido attached to a carbon that also has a heteroatom attached to it.

A “pharmaceutically-acceptable salt” is a cationic salt formed at any acidic (e.g., carboxylic acid) group, or an anionic salt formed at any basic (e.g., amino) group. Many such salts are known in the art, as described in World Patent Publication 87/05297, Johnston et al., published Sep. 11, 1987 incorporated by reference herein. Preferred cationic salts include the alkali metal salts (such as sodium and potassium), and alkaline earth metal salts (such as magnesium and calcium) and organic salts. Preferred anionic salts include the halides (such as chloride salts), sulfonates, carboxylates, phosphates, and the like.

Such salts are well understood by the skilled artisan, and the skilled artisan is able to prepare any number of salts given the knowledge in the art. Furthermore, it is recognized that the skilled artisan may prefer one salt over another for reasons of solubility, stability, formulation ease and the like. Determination and optimization of such salts is within the purview of the skilled artisan's practice.

A “solvate” is a complex formed by the combination of a solute (e.g., a metalloprotease inhibitor) and a solvent (e.g., water). See J. Honig et al., The Van Nostrand Chemist's Dictionary, p. 650 (1953). Pharmaceutically-acceptable solvents used according to this invention include those that do not interfere with the biological activity of the metalloprotease inhibitor (e.g., water, ethanol, acetic acid, N,N-dimethylformamide and others known or readily determined by the skilled artisan).

The terms “optical isomer”, “stereoisomer”, and “diastereomer” have the accepted meanings (see, e.g., Hawley's Condensed Chemical Dictionary, 11th Ed.). The illustration of specific protected forms and other derivatives of the compounds of the instant invention, is not intended to be limiting. The application of other useful protecting groups, salt forms, etc. is within the ability of the skilled artisan.

### DETAILED SUMMARY OF THE INVENTION

The present invention relates generally to a compound having the structure: 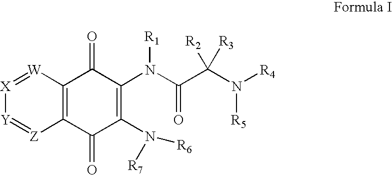
wherein
W, X, Y and Z are each selected from a bond, CH, C—R₈, C—R₉, C—R₁₀, C—R₁₁, O (oxygen), N (nitrogen) and S (sulfur) and no more than two of W, X, Y and Z are simultaneously O, N and S;
   and wherein, R₈, R₉, R₁₀, R₁₁ each may be hydrogen, hydroxyl, sulfhydryl, alkoxy, thioalkoxy, alkyl, halogen, CN, CF₃, NO₂, COOR₁₂, CONR₁₂R₁₃, NR₁₂R₁₃, NR₁₂COR₁₃, NR₁₂SO₂R₁₃, and NR₁₄CONR₁₂R₁₃;
   wherein R₁₂, R₁₃ and R₁₄ are hydrogen, alkyl, heteroalkyl, aryl, arylalkyl, heteroaryl, heteroarylalkyl, cycloalkyl, or heterocycloalkyl;
   NR₁₂R₁₃ may form a substituted or unsubstituted, mono or bicyclic rings, with one to four heteroatoms selected from N, O and S;
   and wherein, R₁₂ and R₁₄ may form a 4, 5, 6 or 7-membered cyclic ring system;
and wherein R₁, R₂, R₃, R₄, and R₅ are each selected from:
   hydrogen, alkyl, substituted or unsubstituted phenyl or polyaromatic rings, substituted or unsubstituted heteroaromatic, with hetero atom(s) as N, O, S, substituted or unsubstituted aralkyl, substituted or unsubstituted cyclo or polycyclo hydrocarbon and mono or polyheterocycle (3-8 atoms per ring) with one to four hetero atoms as N, O, or S; and
   wherein said substitutions are selected from hydroxyl, sulfhydryl, alkoxy, thioalkoxy, alkyl, halogen, CN, CF₃, NO₂, COOR₁₂, CONR₁₂R₁₃, NR₁₂R₁₃, NR₁₂COR₁₃, NR₁₂SO₂R₁₃, and NR₁₄CONR₁₂R₁₃;
      wherein R₁₂, R₁₃ and R₁₄ are each selected from hydrogen, alkyl, heteroalkyl, aryl, arylalkyl, heteroaryl, heteroarylalkyl, cycloalkyl, and heterocycloalkyl;
      NR₁₂R₁₃ is also substituted or unsubstituted, mono or bicyclic rings with one to four heteroatoms selected from N, O and S;
      and wherein R₁₂ and R₁₄ may, in one embodiment, form a 4, 5, 6 or 7-membered cyclic ring system;
and wherein R₁, R₄, R₅, R₆ and R₇ may also be selected from: 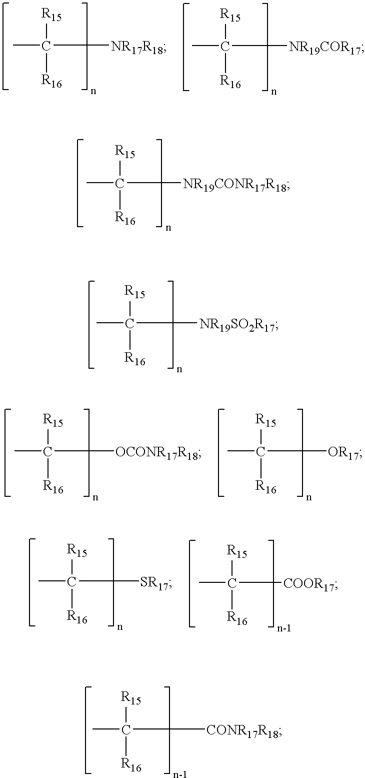
   wherein n is 2, 3 or 4 and R₁₅, R₁₆, R₁₇, R₁₈ and R₁₉ are selected from hydrogen, alkyl, cycloalkyl, unsubstituted or substituted aryl, unsubstituted or substituted heteroaryl, and unsubstituted or substituted alkylaryl;
   and NR₁₇R₁₈ may also be a substituted or unsubstituted, mono or bicyclic ring with one to four heteroatoms selected from N, O and S;
   and wherein R₁₇ and R₁₉ may form a 4, 5, 6 or 7-membered cyclic ring system;
   and wherein R₄ is also selected from —COR₁₃, —SO₂R₁₃, —CONR₁₂R₁₃, and —C(═NR₁₉)NR₁₇R₁₈;
wherein R₆ and R₇ may also each be selected from:
   alkyl, substituted and unsubstituted phenyl or polyaromatic, substituted and unsubstituted heteroaromatic rings with hetero atoms selected from N, O and S, substituted and unsubstituted aralkyl, substituted and unsubstituted, cyclic or polycyclic hydrocarbon and mono or polyheterocyclic rings, each of 3-8 atoms, said heterocycle having one to four hetero atoms selected from N, O and S; and
   wherein substitutions are selected from hydroxyl, sulfhydryl, alkoxy, thioalkoxy, alkyl, halogen, CN, CF₃, NO₂, COOR₁₂, CONR₁₂R₁₃, NR₁₂R₁₃, NR₁₂COR₁₃, NR₁₂SO₂R₁₃, NR₁₄CONR₁₂R₁₃;
      wherein R₁₂, R₁₃ and R₁₄ are each selected from hydrogen, alkyl, heteroalkyl, aryl, arylalkyl, heteroaryl, heteroarylalkyl, cycloalkyl, and heterocycloalkyl;
      NR₁₂R₁₃ is also unsubstituted, monosubstituted or polysubstituted mono or bicyclic ring with one to four heteroatoms such as N, O, S;
         and wherein NR₄R₅ and NR₆R₇ may each form a substituted or unsubstituted, mono or bicyclic ring comprising one to four heteroatoms selected from N, O and S and wherein said N may also be substituted or unsubstituted,
         and including salts of any of the above-recited structures.

In another preferred embodiment, R₁, R₂, R₃, R₄, and R₅, are each selected from hydrogen, alkyl, substituted or unsubstituted phenyl, substituted or unsubstituted polyaromatic, and substituted or unsubstituted heteroaromatic comprising one or more hetero atom(s) selected from N, O and S.

In another preferred embodiment, R₁, R₂, R₃, R₄, and R₅ are each selected from substituted or unsubstituted aralkyl, substituted or unsubstituted cyclo or polycyclo hydrocarbon or mono or polyheterocycle (3-8 atoms per ring) with one to four hetero atoms selected from N, O and S.

In any of these preferred embodimenits, substitutions are selected from hydroxyl, sulfhydryl, lower alkoxy (1-6 carbon), lower thioalkoxy (1-6 carbon), lower alkyl (1-6 carbon), halogen, CN, CF₃, NO₂, COOR₁₂, CONR₁₂R₁₃, NR₁₂R₁₃, NR₁₂COR₁₃, NR₁₂SO₂R₁₃, and NR₁₄CONR₁₂R₁₃, wherein R₁₂, R₁₃ and R₁₄ are hydrogen, alkyl, heteroalkyl, aryl, arylalkyl, heteroaryl, heteroarylalkyl, cycloalkyl, or heterocycloalkyl. In a further preferred embodiment of the foregoing, R₁₂ and R₁₄ form a 4, 5, 6 or 7-member cyclic ring system.

In a further preferred embodiment, NR₁₂R₁₃ forms a substituted or unsubstituted mono or bicyclic ring comprising one to four heteroatoms selected from N, O and S.

In one preferred embodiment, R₁, R₄, R₅, R₆ and R₇ are each selected from: 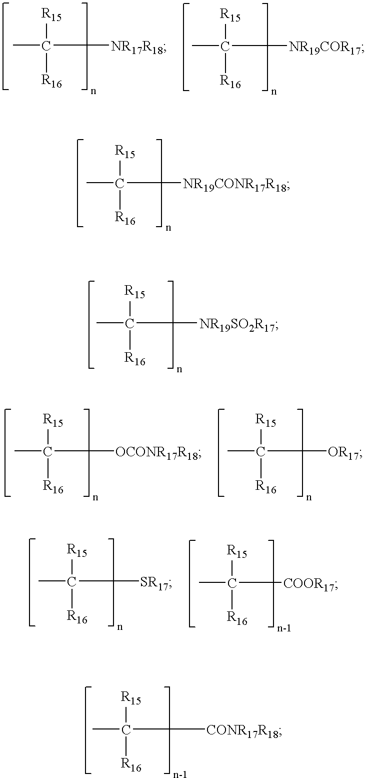
wherein n is 2, 3 or 4 and R₁₅, R₁₆, R₁₇, R₁₈ and R₁₉ are selected from hydrogen, lower alkyl, cycloalkyl, substituted and unsubstituted aryl, substituted or unsubstituted heteroaryl, and substituted or unsubstituted alkylaryl. In a preferred embodiment thereof, NR₁₇R₁₈ is forms a substituted or unsubstituted, mono or bicyclic ring comprising one to four heteroatoms selected from N, O and S. In another preferred embodiment thereof, R₁₇ and R₁₉ form a 4, 5, 6 or 7-membered cyclic ring system.

In a preferred embodiment of the compounds of Formula I, W and Z are each selected from C—R₈, C—R₁₁, and N, and X and Y are each selected from C—R₉ and C—R₉. In another preferred embodiment, X and Y are each selected from C—R₉, C—R₁₀ and N and wherein W and Z are each selected from C—R₈ and C—R₁₁. In another preferred embodiment, W is C—R₈ or N, and X, Y and Z are each selected from C—R₉, C—R₁₀ and C—R₁₁.

Where a position in a structure, such as W, X, Y or Z, or a substituent, such as an R group, as recited above, is described as selected from, it means that each of W, X, Y and Z, or R, can be selected from the indicated group of structures or atoms and each is selected independently of the others unless it is expressly stated herein to be otherwise. By “independent” is meant that the selection of one substituent does not limit the range of selection for another substituent, unless expressly stated as such. For example, where X and Y are selected from a range of atoms, such as N, O and S, then X and Y may be the same or different and the selection of one does not limit the range of the other. Thus, if X is nitrogen then Y can still be N, O or S.

Where a position, for example, in a ring, is described as being selected from “a bond” etc., this means that the position is not occupied by an atom. Thus, if in Formula I, X is a bond, then the ring with W, X, Y and Z is a 5 membered ring instead of a 6 membered ring.

In a preferred embodiment, NR₄R₅ and/or NR₆R₇ of Formula I form(s) a piperazine ring, preferably an N-acetylpiperazinyl group.

In a preferred embodiment, —NR₄R₅ and/or —NR₆R₇ of Formula I is a substituted or unsubstituted morpholinyl group. In a highly preferred embodiment thereof, R₆ and R₇ are both hydrogen. In a most preferred embodiment, R₂ and R₃ are both hydrogen and —NR₄R₅ forms an unsubstituted morpholinyl group.

In a preferred embodiment, NR₄R₅ and/or NR₆R₇ of Formula I is a piperidine ring, preferably a substituted piperidine ring, most preferably 4-hydroxypiperidine.

In a highly preferred embodiment of any of the structures of the present invention, R₁, R₆ and R₇ of Formula I are each methyl.

In another preferred embodiment of the compounds of the invention, Z is C—R₁₀, or N and W, Y and Z are each selected from C—R₈, C—R₉ and C—R₁₁. In one embodiment of the latter, X is C—R₁₀ or N and W, Y and Z are each selected from C—R₈, C—R₉ and C—R₁₁. In a preferred embodiment of the latter Y is C—R₁₀ or N and W, X, and Z are each selected from CH, C—R₈, C—R₉ and C—R₁₁. In a most preferred embodiment thereof, W, X, Y and Z are each selected from CH, C—R₈, C—R₉, C—R₁₀ and C—R₁₁, most preferably where W X, Y and Z are each CH (thereby forming a phenyl ring).

In another preferred embodiment of the compounds of the invention, R₂ and R₃ are selected from hydrogen, lower alkyl (1-6 carbon) or aryl. In a further preferred embodiment of the compounds of the invention, R₁ is selected from hydrogen, alkyl, cycloalkyl, unsubstituted or substituted phenyl, unsubstituted or substituted benzyl, -methylpyridine, -ethylpyridine, -methylindole, -ethylindole, alkoxyethyl-, hydroxyethyl-, N,N-diaikyl-ethyl, N,N-dialkyl-propyl, -methylpyrrole, -ethylpyrrole, -methylfuran, -ethylfuran, -alkylmorpholine, -alkylpiperizine, -alkypiperidine, and -alkylpyrrolidine and wherein R₂ and R₃ are selected from hydrogen, lower alkyl (1-6 carbons) and aryl.

In another preferred embodiment of the compounds of the invention, R₄ and R₅ are each selected from hydrogen, alkyl, cycloalkyl, unsubstituted or substituted phenyl, unsubstituted or substituted benzyl, -methylpyridine, -ethylpyridine, -methylindole, -ethylindole, alkoxyethyl-, hydroxyethyl-, N,N-dialkyl-ethyl-, N,N-dialkyl-propyl-, -methylpyrrole, -ethylpyrrole, -methylfuran, -ethylfuran, -alkyqmorpholine, -alkylpiperizine, -alkypiperidine, and -alkylpyrrolidine, and wherein R₂ and R₃ are selected from hydrogen, lower alkyl (1-6 carbon) and aryl.

In another preferred embodiment of the compounds of the invention, R₆ and R₇ are selected from alkyl, cycloalkyl, unsubstituted or substituted phenyl, unsubstituted or substituted benzyl, -methylpyridine, -ethylpyridine, -methylindole, -ethylindole, alkoxyethyl-, hydroxyethyl-, N,N-dialkyl-ethyl, N,N-dialkyl-propyl, -methylpyrrole, -ethylpyrrole, -methylfuran, -ethylfuran, -alkylmorpholine, -alkylpiperizine, -alkypiperidine, and -alkylpyrrolidine, and R₂ and R₃ are each selected from hydrogen, lower alkyl (1-6 carbons) and aryl.

In other preferred embodiments, R₂ and R₃ are selected from hydrogen, lower alkyl (1-6 carbon) and aryl, wherein R₁, R₄ and R₅ are each selected from hydrogen, alkyl, cycloalkyl, unsubstituted or substituted phenyl, unsubstituted or substituted benzyl, -methylpyridine, -ethylpyridine, -methylindole, -ethylindole, alkoxyethyl-, hydroxyethyl-, N,N-dialkyl-ethyl-, N,N-dialkyl-propyl-, -methylpyrrole, -ethylpyrrole, -methylfuran, -ethylfuran, -alkylmorpholine, -alkylpiperizine, -alkypiperidine, and -alkylpyrrolidine, and wherein R₂ and R₃ are selected from hydrogen, lower alkyl (1-6 carbon) or aryl and wherein R₆ and R₇ are selected from alkyl, cycloalkyl, unsubstituted or substituted phenyl, unsubstituted or substituted benzyl, -methylpyridine, -ethylpyridine, -methylindole, -ethylindole, alkoxyethyl-, hydroxyethyl-, N,N-dialkyl-ethyl, N,N-dialkyl-propyl, -methylpyrrole, -ethylpyrrole, -methylfuran, -ethylfuran, -alkylmorpholine, -alkylpiperizine, -alkypiperidine, and -alkylpyrrolidine.

In another preferred embodiment of the compounds of the invention having Formula 1, R₂ and R₃ are each selected from hydrogen and alkyl, and wherein R₄ and R₆ are each selected from alkyl and 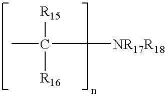
wherein n is 2 ,3 or 4 and wherein one or both of R₅ and R₇ is alkyl, preferably both, and in either case most preferably wherein the alkyl is methyl.

In another preferred embodimentof Formula I, R₁ is alkyl while R₂ and R₃ are each selected from hydrogen and alkyl, and R₄ and R₆ are each selected from alkyl and

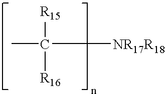

wherein n is 2, 3 or 4 and one or both of R₅ and R₇ is alkyl, preferably both, and in either case most preferably wherein the alkyl is methyl.

In another preferred embodiment of Formula 1, R₂ and R₃ are each selected from hydrogen and alkyl while R₄ and R₆ are each selected from alkyl and 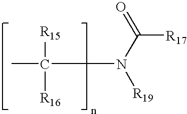
where n is 2, 3 or 4 and one or both of R5 and R7 is alkyl, preferably both, and in either case most preferably wherein the alkyl is methyl.
In another preferred embodiment of Formula 1, R₂ and R₃ are each selected from hydrogen and alkyl, wherein R₄ and R₆ are each selected from alkyl and 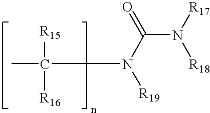
where n is 2, 3 or 4 and at least one of R₅ and R₇ is alkyl, preferably both, and in either case most preferably wherein the alkyl is methyl.

In another preferred embodiment of Formula 1, R₂ and R₃ are each selected from hydrogen and alkyl, and R₄ and R₆ are each selected from alkyl and 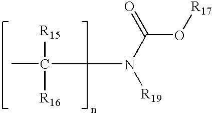
where n is 2, 3 or 4 and at least one of R5 and R7 is alkyl, preferably both, and in either case most preferably wherein the alkyl is methyl.

In another preferred embodiment of Formula 1, R2 and R3 are each selected from hydrogen and alkyl and R4 and R6, are each selected from alkyl and 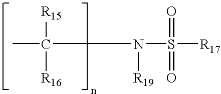
wherein n is 2, 3 or 4 and wherein one or both of R₅ and R₇ is alkyl, preferably both, and in either case most preferably wherein the alkyl is methyl.

In another preferred embodiment of Formula 1, R₂ and R₃ are each be hydrogen or alkyl, R₄ and R₆ are each selected from alkyl and 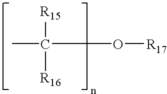
where n is 2, 3 or 4 and one or both of R₅ and R₇ is alkyl, and in either case most preferably wherein the alkyl is methyl.

In separate embodiments, the present invention encompasses compounds having a structure found in Table 1 including salts thereof, a compound having a structure of Table 2 including salts thereof, a compound having a structure of Table 3 including salts thereof, a compound having a structure of Table 4 including salts thereof, a compound having a structure of Table 5 including salts thereof, a compound having a structure of Table 6 including salts thereof, a compound having a structure of Table 7 including salts thereof, a compound having a structure of Table 8 including salts thereof, a compound having a structure of Table 9 including salts thereof, a compound having a structure of Table 11 including salts thereof, a compound having a structure of Table 12 including salts thereof, a compound having a structure of Table 13 including salts thereof, a compound having a structure of Table 14 including salts thereof, a compound having a structure of Table 15 including salts thereof, a compound having a structure of Table 16 including salts thereof, a compound having a structure of Table 17 including salts thereof, and a compound having a structure of Table 18 including salts thereof, and in each case most preferably pharmaceutically acceptable salts thereof. It is to be understood that each of the structures defined in each of these tables is considered to be a separate and preferred embodiment of the present invention.

In another aspect, the present invention relates to compositions of any of the compounds of the invention, preferably wherein such compound is present in a pharmaceutically acceptable carrier and in a therapeutically effective amount. Such compositions will generally comprise an amount of such compound that is not toxic (i.e., an amount that is safe for therapeutic uses).

In accordance with the foregoing, the present invention is directed to use of the compounds of the invention as active ingredients for medicaments, in particular for medicaments useful for the treatment of tumors. The compounds of the invention will thus be present in pharmaceutical compositions containing compounds of formula I as active ingredients, in admixture with pharmaceutically acceptable vehicles and excipients, which includes any pharmaceutical agent that does not itself induce the production of antibodies harmful to the individual receiving the composition, and which may be administered without undue toxicity. Pharmaceutically acceptable carriers include, but are not limited to, liquids such as water, saline, glycerol and ethanol, and the like, including carriers useful in forming sprays for nasal and other respiratory tract delivery, or for delivery, to the ophthalmic system. A thorough discussion of pharmaceutically acceptable carriers, diluents, and other excipients is presented in REMINGTON'S PHARMACEUTICAL SCIENCES (Mack Pub. Co., N.J. current edition). Use of such carriers is well known to those skilled in the art and will not be discussed further herein.

Also in accordance with the foregoing, the present invention relates to a method for preventing or treating a disease associated with a change in levels of expression of particular sets of genes in a mammal, comprising administering to said mammal an effective amount of a compound of the invention.

In another aspect, the present invention relates to a method for preventing or treating a disorder modulated by altered gene expression, wherein the disorder is selected from the group consisting of cancer, cardiovascular disorders, arthritis, osteoporosis, inflammation, periodontal disease and skin disorders, comprising administering to a mammal in need of such treatment or prevention a therapeutically effective amount of a compound of the invention.

In a preferred embodiment thereof, the disorder is cancer, more preferably colon cancer, most preferably adenocarcinoma, and the treatment prevents, arrests or reverts tumor growth, metastasis or both.

The compounds of the invention will commonly exert a therapeutic effect by modulation of one or more genes found in a cell, especially a mammalian cell, such as a cancer cell, preferably colon cancer and most preferably adenocarcinoma. Thus, a compound, or compounds, of the invention can be used to determine or demarcate a set of genes by determining modulation of such set of genes by one or more compounds of the invention. For example, where a set of genes is found to be up-regulated in cancer cells versus otherwise normal cells, especially normal cells of the same tissue or organ as the cancer cells, a set of genes can be determined by their common property of being modulated (based on a change in expression of the genes, such as a change in rate or amount of RNA transcribed or the amount of polypeptide produced by said expression) by contacting such genes, or a cell containing such genes, with one or more of the compounds of the invention. The extent of such modulation may, of course, be related to the amount of said compound, or compounds, used in the contacting. Such modulation may include the increased expression of all the determined genes (i.e., the genes of the set), the decreased expression of all genes of the set, or the increase in expression of some of the genes of the set and decreased expression of others. Thus, a gene not modulated by the test compound (the compound used in contacting the genes or cell containing them) is not considered a member of the set.

Thus, the present invention relates to a gene set wherein expression of each member of said gene set is modulated as a result of contacting said gene set with a compound of the invention. In specific embodiments, expression of each member of said gene set is increased as a result of said contacting or is decreased as a result of said contacting. In another preferred embodiment, the gene set is present in a cell. Such a gene set will commonly be related to a specific disease process, such as a set of genes all of which are modulated by a compound of the invention wherein such compound has a specific therapeutic effect, such as being an anti-neoplastic agent.

In another aspect, the present invention relates to a method for identifying an agent that modulates the expression of a gene set of the invention, comprising:
(a) contacting, or otherwise using, a compound, such as a test compound, a test system, such as a source of genes or polynucleotides, for example, those found to be related to a given disease or disorder, or a set that is modulated by a given compound, or group of compounds, especially where these are found in a cell, so that the cell represents the test system, containing one or more polynucleotides corresponding to each of the members of the gene set of the invention under conditions wherein the members of said gene set are being expressed;
(b) determining a change in expression of each of said one or more polynucleotides of step (a) as a result of said treatment;
wherein said change in expression of step (b) indicates modulation of the members of said gene set by the test compound thereby identifying a test compound that modulates the expression of said gene set.

In one embodiment, the cell is a naturally derived cell that contains genes of a gene set or may be a recombinant cell engineered to comprise the genes or polynucleotides of the gene set. In an alternative embodiment, the test system may comprise the genes or polynucleotides in a cell-free system.

In a related aspect, the present invention provides a method for identifying a test compound that modulates the expression of a gene set, such as a gene set of the invention, comprising:
(a) contacting a test compound with one or more polynucleotides corresponding to each of the members of the gene set of the invention under conditions wherein the members of said gene set are being expressed;
(b) determining a change in expression of each of said one or more polynucleotides of step (a) as a result of said contacting;
wherein said change in expression of step (b) indicates modulation of the members of said gene set thereby identifying a test compound that modulates the expression of said gene set.

As used herein, “corresponding genes” or “corresponding polynucleotides” or “polynucleotides corresponding to genes” refers to polynucleotides and/or genes that encode an RNA that is at least 90% identical, preferably at least 95% identical, most preferably at least 98% identical, and especially identical, to an RNA encoded by one of the genes disclosed herein in Table 19. Such genes will also encode the same polypeptide sequence, but may include differences in such amino acid sequences where such differences are limited to conservative amino acid substitutions, such as where the same overall three dimensional structure, is maintained. A “corresponding gene” includes splice variants thereof.

Because a polynucleotide or gene used in the methods of the invention “corresponds to” a gene present in one of the gene sets of the invention, such as genes identified in Table 19, such polynucleotide or gene encodes an RNA (processed or unprocessed, including naturally occurring splice variants and alleles) that is at least 90% identical, preferably at least 95% identical, most preferably at least 98% identical to, and especially identical to, an RNA that would be encoded by, or be complementary to, such as by hybridization with, a gene of Table 19, or genes of any gene set identified according to the invention. Polynucleotides encoding the same proteins as any of these genes, regardless of the percent identity of the sequences of such genes, and/or polynucleotides, are also specifically contemplated by any of the methods of the present invention. The polynucleotides used in the methods of the invention also include any open reading frames, as defined herein, present therein. As used herein, the term “open reading frame” (or ORF) means a series of triplets coding for amino acids without any termination codons and is a sequence (potentially) translatable into protein.

The polynucleotides useful in the methods of the invention may be genomic in nature and thus represent the sequence of an actual gene, such as a human gene, or may be a cDNA sequence derived from a messenger RNA (mRNA) and thus represent contiguous exonic sequences derived from a corresponding genomic sequence, or they may be wholly synthetic in origin for purposes of practicing the processes of the invention. Because of the processing that may take place in transforming the initial RNA transcript into the final mRNA, the sequences disclosed herein may represent less than the full genomic sequence. They may also represent sequences derived from ribosomal and transfer RNAs. Consequently, the gene as present in the cell (and representing the genomic sequence) and the polynucleotide transcripts disclosed herein, including cDNA sequences, may be identical or may be such that the cDNAs contain less than the full genomic sequence. Such genes and cDNA sequences are still considered “corresponding sequences” (as defined elsewhere herein) because they both encode the same or related RNA sequences (i.e., related in the sense of being splice variants or RNAs at different stages of processing). Thus, by way of non-limiting example only, a gene that encodes an RNA transcript, which is then processed into a shorter mRNA, is deemed to encode both such RNAs and therefore encqdes an RNA complementary to (using the usual Watson-Crick complementarity rules), or that would otherwise be encoded by, a cDNA (for example, a sequence as disclosed herein). Thus, the sequences disclosed herein correspond to genes contained in the cancerous cells (here, breast cancer) and are used to determine gene, activity or expression because they represent the same sequence or are complementary to RNAs encoded by the gene. Such a gene also includes different alleles and splice variants that may occur in the cells used in the methods of the invention, such as where recombinant cells are used to assay for anti-neoplastic agents and such cells have been engineered to express a polynucleotide as disclosed herein, including cells that have been engineered to express such polynucleotides at a higher level than is found in non-engineered cancerous cells or where such recombinant cells express such polynucleotides only after having been engineered to do so. Such engineering includes genetic engineering, such as where one or more of the polynucleotides disclosed herein has been inserted into the genome of such cell or is present in a vector.

Such cells, especially mammalian cells, may also be engineered to express on their surfaces one or more of the polypeptides of the invention for testing with antibodies or other agents capable of masking such polypeptides and thereby removing the cancerous nature of the cell. Such engineering includes both genetic engineering, where, the genetic complement of the cells is engineered to express the, polypeptide, as well as non-genetic engineering, whereby the cell has been physically manipulated to incorporate a polypeptide of the invention in its plasma membrane, such asby direct insertion using chemical and/or other agents to achieve this result.

In a preferred embodiment of such method, the determined change in expression is a decrease in expression of said one or more polynucleotides or a decrease in said expression. In other preferred embodiments, the determined change in expression is a change in transcription of said one or more polynucleotides or a change in activity of a polypeptide, or expression product, encoded by said polynucleotide, including a change in the amount of said polypeptide synthesized, such as by a cell. The term “expression product” means that polypeptide or protein that is the natural translation product of the gene and any nucleic acid sequence coding equivalents resulting from genetic code degeneracy and thus coding for the same amino acid(s).

In additional preferred embodiments, said one or more polynucleotides are present in a cell, preferably a cancer cell, more preferably a colon and breast cancer cell, and most preferably where the coloncancer cell is an adenocarcinoma cancer cell. In another preferred embodiment of the invention, the cell is a recombinant cell engineered to contain said set of genes.

Such methods serve to identify other compounds that have like activity, including expected therapeutic activity, as the compounds of the invention and thus serve as the basis for large scale screening assays for therapeutic compounds. As a result, one or more compounds of the invention can be utilized to determine the presents of gene sets and subsets within the genome of a cell. Thus, the set of all genes modulated by a group of structurally related compounds of the invention can form a gene set while the different sets of genes regulated by each compound of a group will form a subset. By way of non-limiting example, where a structurally related group of 5 of the compounds of the invention (all having generally the structure of Formula I) modulate (by increasing or decreasing) expression of determined genes 1-20, this latter group of genes forms a gene set. Further examination then determines that genes 1-6 are modulated by compound A, genes 7-10 are modulated by compound B, genes 2-4 and 9-12 are modulated by compound C, genes 10-20 are modulated by compound D and the even numbered genes are modulated by compound E. Each of these groups of genes, such as the genes modulated by compound C, is considered a subset of the gene set of genes 1-20. In an analogous manner, the genes modulated by compound E can be themselves further subdivided into at least 2 subsets wherein one subset is made up of the genes whose expression is increased by compound E while the other subset is made up of genes whose expression is decreased by compound E, thus yielding subsets of subsets. It should be noted that within the context of the present invention, it is not necessary to identify subsets and that each so-called subset is, in its own right, a gene set as used in the invention. The identification of sets and subsets is thus a function of the extent that a user of the methods of the invention wishes to determine modulation of genes resulting from contacting of one or more compounds of the invention. Thus, the genes modulated by a single compound form a gene set and it is not necessary, in carrying out the methods of the invention, to compare different groups of genes for modulation by more than one compound but this may, of course, be done.

In accordance with the foregoing, the present invention relates to a set of genes comprising a plurality of subsets of genes wherein each subset of said plurality is a gene set identified by the methods of the invention. The present invention also relates to compounds identified as having activity using the methods of the invention, such as novel compounds not specifically described herein by structure but which have been identified by their ability to modulates one or more gene sets modulated by compounds of the invention.

In a preferred embodiment, the present invention encompasses the gene sets and subsets of the genes identified in Table 19.

The present invention comprises also processes for the preparation of compounds of formula I, and the relative key intermediates

Comp und Pr paration

The compounds of the invention can be prepared using a variety of procedures known in the art. The starting materials used in preparing the compounds of the invention are known, made by known methods, or are commercially available. Particularly preferred syntheses are described in the following general reaction schemes;

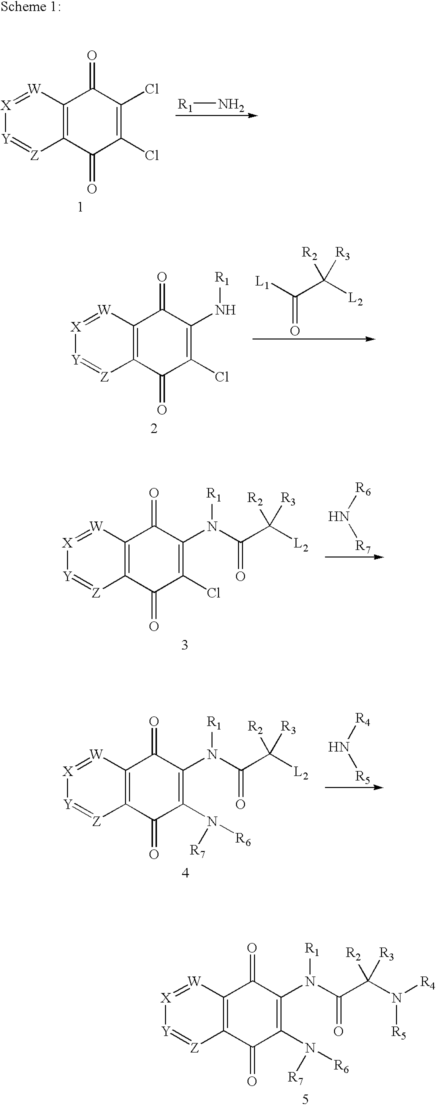

The dichloro compound 1 is either commercially available or can be synthesized using methods known in the literature.
1. Shaikh I. A. et al, J. Med. Chem, 29(8), 1329-1340, (1986)
2. Vlderrama el al, Syn. Comm., 27(12), 2143-2157, (1997)
3. Chu, Kwong-Yung; et al. Journal of the Chemical Society, Perkin Transactions 1: Organic and Bio-Organic Chemistry (1972-1999) (1978)
4. Matsuhisa A. et al, Patent WO 01/60803 A1

The compound 1 is reacted with an amine in an appropriate solvent to provide the corresponding derivative 2. The compound 2 is then reacted with an appropriate 2-halo, 2-substituted acetyl halide to obtain the corresponding 3 derivatives. A reaction of crude or purified compound 3 with an amine gives compound 4. Compound 4 with or without isolation is treated with an amine in a suitable solvent at an appropriate temperature to afford compound 5.

In the same way, independent and selective modification of R₁, R₂, R₃, R₄, R₅, R₆, and R₇ using methods known in the literature readily affords additional compounds of formula I. Thus, compounds for which no separate preparation is provided herein are made by methods known in the literature or are of common knowledge to the skilled artisan.

The skilled artisan will recognize that some reactions are best carried out when another potentially reactive functionality on the molecule is masked or protected, thus avoiding any undesirable side reactions and/or increasing the yield of the reaction. Often protecting groups are used to accomplish such increased yields or to avoid the undesired reactions. Such reactions are well within the ability of the skilled artisan. Some examples are found in T. Greene, Protecting Groups in Organic Synthesis.

In addition, it is to be appreciated that one optical isomer may have favorable properties over the other and thus the disclosure of a racemic mixture within the present invention may also include either optically active isomer if such isomer has advantageous physiological activity in accordance with the methods of the invention.

### EXAMPLE-A1

2-Chloro-3-methylamino-[1,4]naphthoquinone

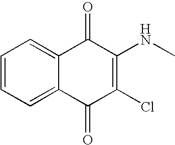

To a solution of 22.7 g (100 mmol, 1 equivalent) of 2,3-dichloro-[1,4]naphthopquinone in 350 ml of anhydrous THF was added 200 ml of 2.0M methyl amine in THF (200 mmol, 2 equivalents). To the mixture was added 34 ml of N, N-diisopropylethylamine (200 mmol, 2 equivalents) and it was shaken at room temperature for overnight (16-20 hours).

The red precipitates formed were filtered and washed with ether. The residue was again washed with water and ether. The solid was dried under vacuum. The filtrate was checked for the desired product, and then THF was evaporated. The residue was recrystallized with dichlorotnethane/ether. The titled compound was collected as a red solid (18 g, Yield 74%).

In a process analogous to Example A1 using appropriate starting materials, the corresponding compounds are prepared as follows:

| Example | Chemical Name |
|---|---|
| A2 | (3-Chloro-1,4-dioxo-1,4-dihydro-naphthalen-2- |
| | ylamino)-acetic acid tert-butyl ester |
| A3 | 2-(1-Benzyl-piperidin-4-ylamino)-3-chloro- |
| | [1,4]naphthoquinone |
| A4 | 2-(3-Chloro-1,4-dioxo-1,4-dihydro-naphthalen- |
| | 2-ylamino)-3-phenyl-propionic acid tert-butyl |
| | ester |
| A5 | 2-(4-Acetyl-phenylamino)-3-chloro- |
| | [1,4]naphthoquinone |
| A6 | 2,6-Dichloro-5,8-dihydroxy-3-(3-{4-[3- |
| | (6-oxo-6H-2,10b-diaza-aceanthrylen-5-ylamino)- |
| | propyl]-piperazin-1-yl}-propylamino)- |
| | [1,4]naphthoquinone |
| A7 | 2-Chloro-3-(2-pyridin-4-yl-ethylamino)- |
| | [1,4]naphthoquinone |
| A8 | 2-Chloro-3-(3-{4-[3-(6-oxo-6H-2,10b- |
| | diaza-aceanthrylen-5-ylamino)-propyl]- |
| | piperazin-1-yl}-propylamino)- |
| | [1,4]naphthoquinone |
| A9 | 2-Chloro-3-(3-morpholin-4-yl-propylamino)- |
| | [1,4]naphthoquinone |
| A10 | 2-Chloro-3-(4-dimethylamino-benzylamino)- |
| | [1,4]naphthoquinone |
| A11 | 2-Chloro-3-(4-dimethylamino-phenylamino)- |
| | [1,4]naphthoquinone |
| A12 | 2-Chloro-3-[(1-ethyl-pyrrolidin-2-ylmethyl)- |
| | amino]-[1,4]naphthoquinone |
| A13 | 2-Chloro-3-[2-(1,2,2,6,6-pentamethyl- |
| | piperidin-4-yl)-ethylamino]- |
| | [1,4]naphthoquinone |
| A14 | 2-Chloro-3-[3-(2-oxo-pyrrolidin-1-yl)- |
| | propylamino]-[1,4]naphthoquinone |
| A15 | 2-Chloro-3-[3-(methyl-phenyl-amino)- |
| | propylamino]-[1,4]naphthoquinone |
| A16 | 2-Chloro-3-{[(4-methyl-pyridin-2-yl)- |
| | phenyl-methyl]-amino}- |
| | [1,4]naphthoquinone |
| A17 | 2-Chloro-3-phenylamino-[1,4]naphthoquinone |
| A18 | 2-Chloro-5,8-dihydroxy-3-(3-{4-[3-(6- |
| | oxo-6H-2,10b-diaza-aceanthrylen-5-ylamino)- |
| | propyl]-piperazin-1-yl}-propylamino)- |
| | [1,4]naphthoquinone |
| A19 | 4-(3-Chloro-1,4-dioxo-1,4-dihydro-naphthalen- |
| | 2-ylamino)-benzoic acid ethyl ester |

### EXAMPLE-B1

2-Bromo-N-(3-chloro-1,4-dioxo-1,4-dihydro-naphthalen-2-yl)-N-methyl-acetamide

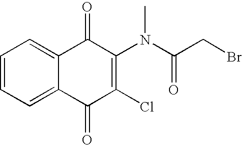

To a solution of 8 g of 2-chloro-3-methylamino-[1,4]naphthoquinone (36 mmol) in 400 ml 1,4-dioxane was added 10 g of potassium carbonate (72 mmol). The mixture was heated until the starting material was completely dissolved. To the solution, 12.5 ml of bromoacetyl bromide (144 mmol) was added and refluxed for 1 hour. Inorganic materials were filtered and washed thoroughly with dichloromethane. The filtrate was evaporated and the residue was purified by flash silica gel column using 75:25-hexanes: ethyl acetate. The compound was collected as yellow oil. (10 g, Yield 80%).

In a process analogous to Example B1 using appropriate 2-chloro-3-substituted amino [1,4] naphthoquinone (Example A) and corresponding acid bromide following compounds are prepared.
2-Bromo-N-(3-chloro-1,4-dioxo-1,4-dihydro-naphthalen-2-yl)-acetamide
2-Bromo-N-(3-chloro-1,4-dioxo-1,4-dihydro-naphthalen-2-yl)-N-methyl-acetamide
2-Bromo-N-(3-chloro-1,4-dioxo-1,4-dihydro-naphthalen-2-yl)-N-methyl-propionamide

### EXAMPLE 1 (COMPOUND 1, TABLE 1)

2-Dimethylamino-N-(3-dimethylamino-1,4-dioxo-1,4-dihydro-naphthalen-2-yl)-N-methyl-acetamide

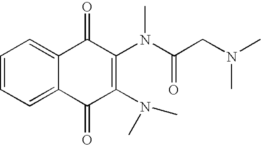

To a solution of 2.5 g of 2-bromo-N-(3-chloro-1,4-dioxo-1,4-dihydro-naphthalen-2-yl)-N-methyl-acetamide (7mmol, 1 equivalent) in 200 ml of ethyl acetate was added 28 ml of 2.0M dimethylamine solution in tetrahydrofuran (56 mmol, 8 equivalents). The amine solution was added in two portions stirring for 15 min after each addition. The solvent was then evaporated and then sample was purified on a silica gel column using initially ethyl acetate and then 10-20% methanol in ethyl acetate. The solvent was evaporated and the residue was dissolved in DMSO. It was then purified further on preparative LCMS using 0.1% NH₄OH in water/acetonitrile as mobile phase. (592 mg, Yield 26%); H¹ NMR (400 MHz, CDCl₃) 2.97 (s, 6H), 3.08 (s, 3H), 3.20 (s, 6H), 3.64 (s, 2H), 7.62 (m, 2H), 7.95 (t, 2H).

Compound 2-119 (Table 1)

In a process analogous to Example 1 (Table 1) using appropriate chloro-bromo naphthoquinone (Example B) and the corresponding secondary amine, following compounds are prepared as shown in Table 1.

### EXAMPLE-C1

2-Chloro-N-(3-chloro-1,4-dioxo-1,4-dihydro-naphthalen-2-yl)-N-methyl-acetamide

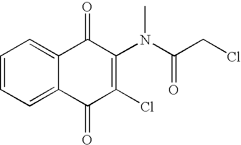

To a solution of 10 g of 2-chloro-3-methylamino-[1,4]naphthoquinone (45 mmol) in 250 mL of dioxane was added 172 mL of chloroacetyl chloride (48 equivalents). The reaction was heated at 85° C. for 16 hours. The solvent was evaporated and the material was purified on silica gel using DCM and hexanes as solvents. The pure fractions were combined and the solvent was evaporated. The product was collected as a yellow/brown solid. (12.1 g, Yield 90%).

In a process analogous to Example C1 using appropriate 2-chloro-3-substituted amino-[1,4 ]naphthoquinone (Example A) and corresponding acid chloride following compounds are prepared.

| Example | Chemical Name |
|---|---|
| C2 | 2-Chloro-N-(3-chloro-1,4-dioxo-1,4-dihydro-naphthalen-2-yl)- |
| | acetamide |
| C3 | 2-Chloro-N-(3-chloro-1,4-dioxo-1,4-dihydro-naphthalen-2-yl)- |
| | propionamide |
| C4 | 2-Chloro-N-(3-chloro-1,4-dioxo-1,4-dihydro-naphthalen-2-yl)- |
| | 2-phenyl-acetamide |
| C5 | 2-Chloro-N-(3-chloro-1,4-dioxo-1,4-dihydro-naphthalen-2-yl)- |
| | N-methyl-propionamide |
| C6 | 2-Chloro-N-(3-chloro-1,4-dioxo-1,4-dihydro-naphthalen-2-yl)- |
| | N-methyl-2-phenyl-acetamide |

### EXAMPLE D1

2-Chloro-N-(3-dimethylamino-1,4-dioxo-1,4-dihydro-naphthalen-2-yl)-N-methyl-acetamide

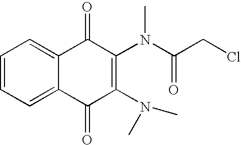

To a solution of 19 g of 2-chloro-N-(3-chloro-1,4-dioxo-1,4-dihydro-naphthalen-2-yl)-N-methyl-acetamide (63 mmol) in 200 mL of ethyl acetate was added slowly 22 mL of N, N-diisopropylethylamine (2 equivalents). 70 mL of 2.0M solution of dimethylamine in terahydrofuran (2.25 equivalents) was diluted with 100 mL of ethyl acetate. This amine solution was added slowly to the reaction mixture over one hour at room temperature. After stirring for an additional hour, the reaction was filtered and the solid material was washed with ethyl acetate. The filtrate was concentrated and purified using a normal phase column chromatography, and ethyl acetate and hexanes as solvents. The pure fractions were combined and the solvent was evaporated. The product was collected as a red solid. (10.1 g, Yield-52%). H¹ NMR (400 MHz, CDCl3): 3.09 (s, 3H), 3.23 (s, 6H), 4.01 (q, 2H), 7.65-7.77 (m, 2H), 8.03 (d, 1H), 8.08 (d, 1H).

In a process analogous to Example D1 using appropriate dichloro naphthoquinone derivatives (Example C) and corresponding secondary amine, the following compounds are prepared.

| Example | Chemical Name |
|---|---|
| D2 | 2-Chloro-N-(3-dimethylamino-1,4-dioxo-1,4-dihydro- |
| | naphthalen-2-yl)-acetamide |
| D3 | 2-Chloro-N-(3-dimethylamino-1,4-dioxo-1,4-dihydro- |
| | naphthalen-2-yl)-propionamide |
| D4 | 2-Chloro-N-(3-dimethylamino-1,4-dioxo-1,4-dihydro- |
| | naphthalen-2-yl)-2-phenyl-acetamide |
| D5 | 2-Chloro-N-(3-dimethylamino-1,4-dioxo-1,4-dihydro- |
| | naphthalen-2-yl)-N-methyl-propionamide |
| D6 | 2-Chloro-N-(3-dimethylamino-1,4-dioxo-1,4-dihydro- |
| | naphthalen-2-yl)-N-methyl-2-phenyl-acetamide |

### EXAMPLE 2 (COMPOUND 1, TABLE 2)

2-Diethylamino-N-(3-dimethylamino-1,4-dioxo-1,4-dihydro-naphthalen-2-yl)-N-methyl-acetamide

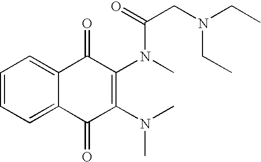

To a solution of 0.54 g of 2-chloro-N-(3-dimethylamino-1,4-dioxo-1,4-dihydro -naphthalen-2-yl)-N-methyl-acetamide (1.8 mmol) in 20 mL of ethyl acetate was added 2.2 mL of ethylamine (21.6 mmol, 12 equiv). The mixture was stirred at room temperature for two hours. The reaction mixture was then filtered and the solid was washed with ethyl acetate until all red material was dissolved. The red filtrate was concentrated and purified on a normal phase column chromatography using ethyl acetate. The pure fractions were combined and concentrated. The solid was then dissolved in 20 mL of DCM and 12 equiv of 1.0M HCl in diethyl ether was added to produce hydrochloride salt. Organic solvents were evaporated and the product was dissolved in 5.0 mL of HPLC grade water. This material was freeze dried to give 0.42 g of final product as its hydrochloride salt. (Yield 62%). H¹ NMR (400 MHz, DMSO, D20) 1.14 (t, 6H), 2.97 (s, 3H), 3.08-3.0 (m, 10H), 3.80 (d, 1H), 4.02 (d, 1H), 7.7-7.9 (m, 2H), 7.89-8.0 (m, 2H).

Compounds 2-119 (Table 2)

In a process analogous to Example 2 using appropriate chloro naphthoquinone (Example D) and the corresponding secondary amine, following compounds are prepared as shown in Table 2.

TABLE 1

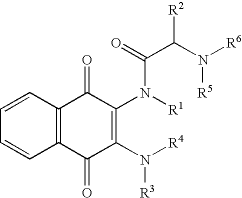

| Cmpd | R₁ | R₂ | 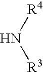 | 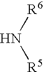 | MW |
|---|---|---|---|---|---|
| 1 | CH3 | H | 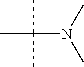 | 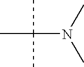 | 315.37 |
| 2 | H | H | 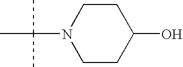 | 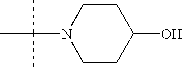 | 413.47 |
| 3 | H | CH3 | 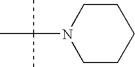 | 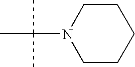 | 395.50 |
| 4 | H | CH3 | 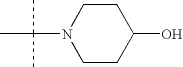 | 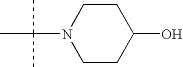 | 427.50 |
| 5 | H | H | 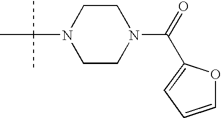 | 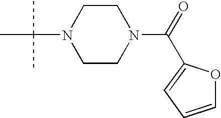 | 571.59 |
| 6 | H | H | 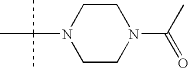 | 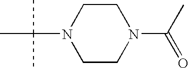 | 467.52 |
| 7 | H | CH3 | 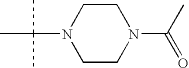 | 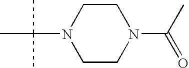 | 481.55 |
| 8 | H | CH3 | 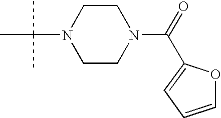 | 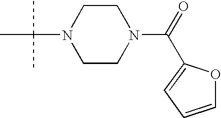 | 585.62 |
| 9 | CH3 | H | 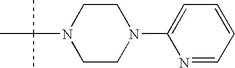 | 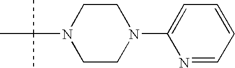 | 551.65 |
| 10 | CH3 | H | 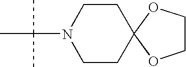 | 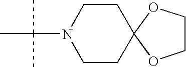 | 511.57 |
| 11 | CH3 | H | 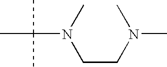 | 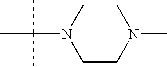 | 429.56 |
| 12 | CH3 | H | 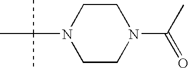 | 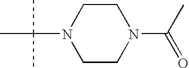 | 481.55 |
| 13 | CH3 | H | 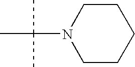 | 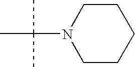 | 395.50 |
| 14 | CH3 | H | 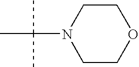 | 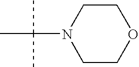 | 399.45 |
| 15 | CH3 | H | 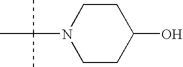 | 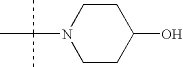 | 427.50 |
| 16 | CH3 | H | 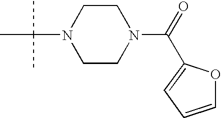 | 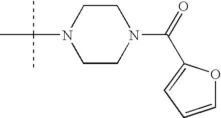 | 585.62 |
| 17 | CH3 | H | 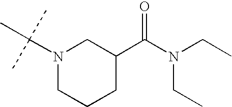 | 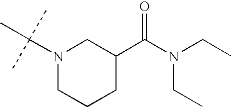 | 593.76 |
| 18 | CH3 | H | 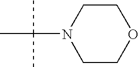 | 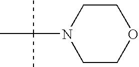 | 399.43 |
| 19 | CH3 | H | 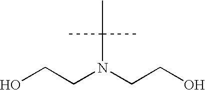 | 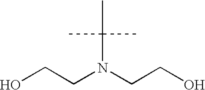 | 435.47 |
| 20 | CH3 | H | 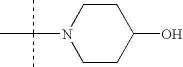 | 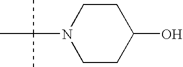 | 427.49 |
| 21 | CH3 | H | 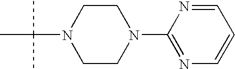 | 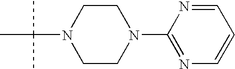 | 553.61 |
| 22 | CH3 | H | 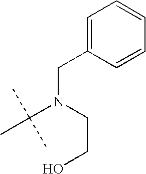 | 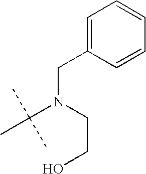 | 527.61 |
| 23 | CH3 | H | 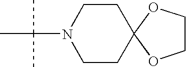 | 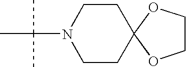 | 511.56 |
| 24 | CH3 | H | 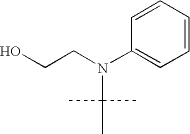 | 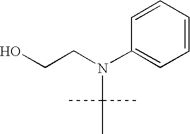 | 499.55 |
| 25 | CH3 | H | 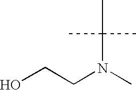 | 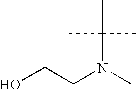 | 375.41 |
| 26 | CH3 | H | 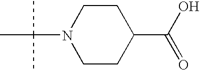 | 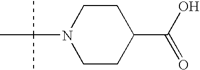 | 483.51 |
| 27 | CH3 | H | 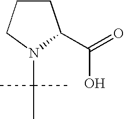 | 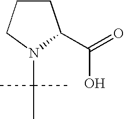 | 455.46 |
| 28 | CH3 | H | 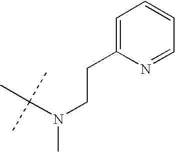 | 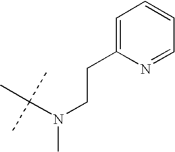 | 497.58 |
| 29 | CH3 | H | 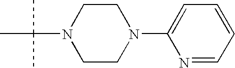 | 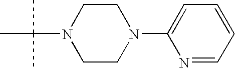 | 551.64 |
| 30 | CH3 | H | 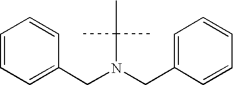 | 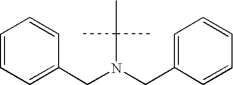 | 619.75 |
| 31 | CH3 | H | 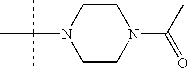 | 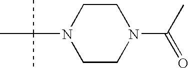 | 481.54 |
| 32 | CH3 | H | 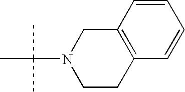 | 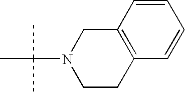 | 491.58 |
| 33 | CH3 | H | 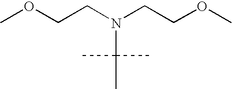 | 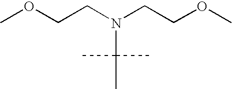 | 491.57 |
| 34 | CH3 | H | 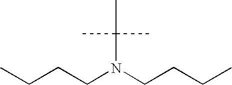 | 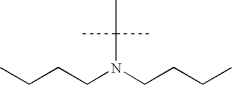 | 483.68 |
| 35 | CH3 | H | 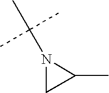 | 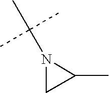 | 339.38 |
| 36 | CH3 | H | 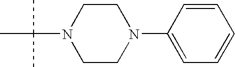 | 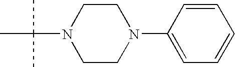 | 549.66 |
| 37 | CH3 | H | 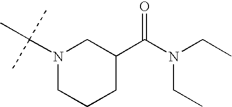 | 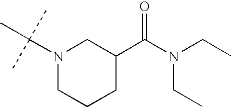 | 593.75 |
| 38 | CH3 | H | 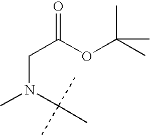 | 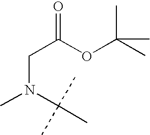 | 515.59 |
| 39 | CH3 | H | 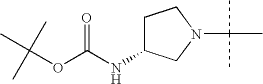 | 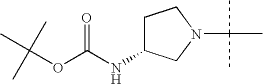 | 597.70 |
| 40 | CH3 | H | 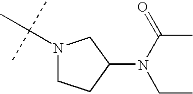 | 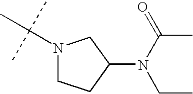 | 537.65 |
| 41 | CH3 | H | 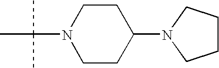 | 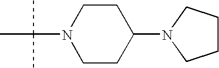 | 533.70 |
| 42 | CH3 | H | 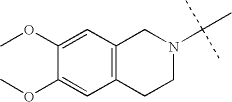 | 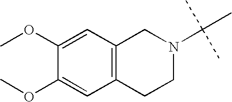 | 611.68 |
| 43 | CH3 | H | 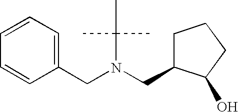 | 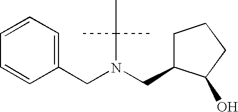 | 635.79 |
| 44 | CH3 | H | 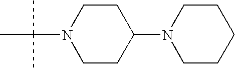 | 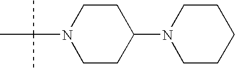 | 561.76 |
| 45 | CH3 | H | 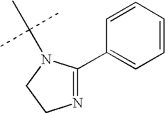 | 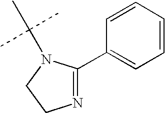 | 517.58 |
| 46 | CH3 | H | 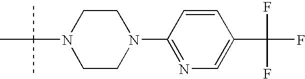 | 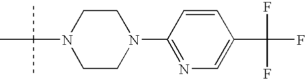 | 687.63 |
| 47 | CH3 | H | 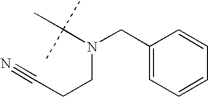 | 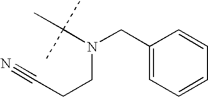 | 545.63 |
| 48 | CH3 | H | 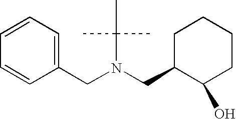 | 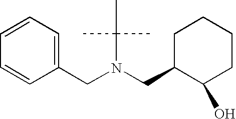 | 663.84 |
| 49 | CH3 | H | 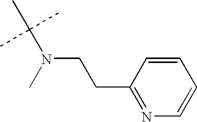 | 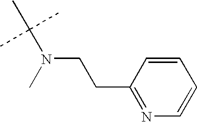 | 497.58 |
| 50 | CH3 | H | 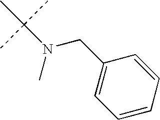 | 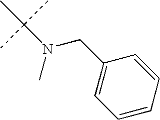 | 467.56 |
| 51 | CH3 | H | 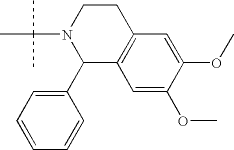 | 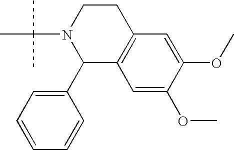 | 763.88 |
| 52 | CH3 | H | 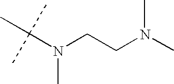 | 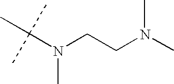 | 429.56 |
| 53 | CH3 | H | 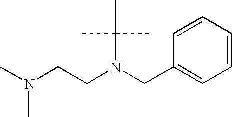 | 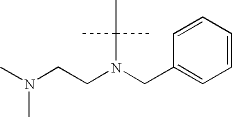 | 581.75 |
| 54 | CH3 | H | 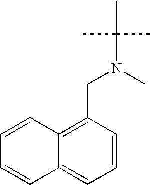 | 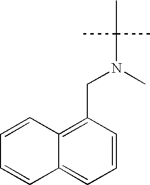 | 567.68 |
| 55 | CH3 | H | 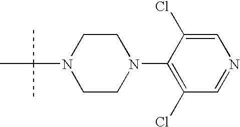 | 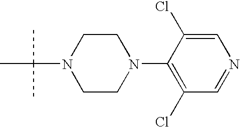 | 689.42 |
| 56 | CH3 | H | 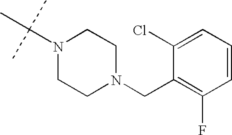 | 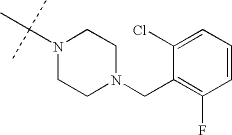 | 682.59 |
| 57 | CH3 | H | 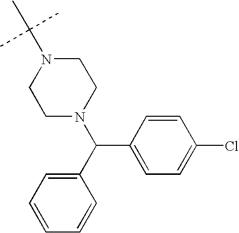 | 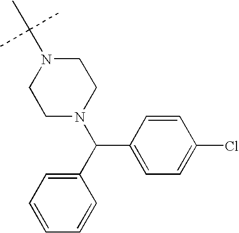 | 798.80 |
| 58 | CH3 | H | 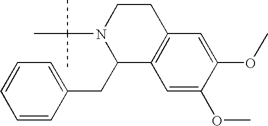 | 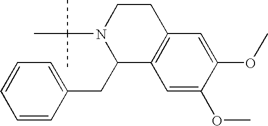 | 791.93 |
| 59 | CH3 | H | 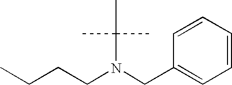 | 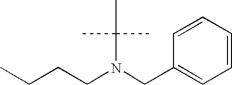 | 551.72 |
| 60 | CH3 | H | 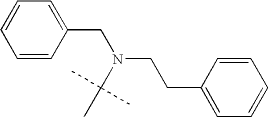 | 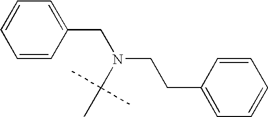 | 647.80 |
| 61 | CH3 | H | 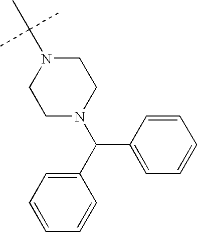 | 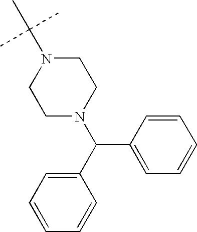 | 729.91 |
| 62 | CH3 | H | 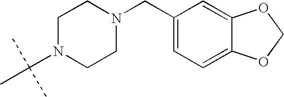 | 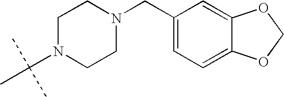 | 665.73 |
| 63 | CH3 | H | 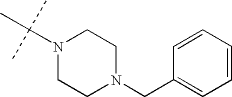 | 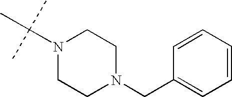 | 577.71 |
| 64 | CH3 | H | 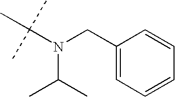 | 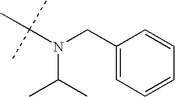 | 523.66 |
| 65 | CH3 | H | 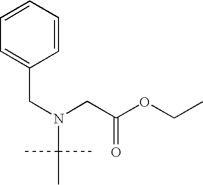 | 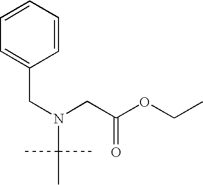 | 611.68 |
| 66 | CH3 | H | 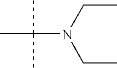 | 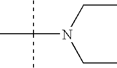 | 371.47 |
| 67 | CH3 | H | 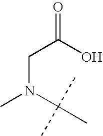 | 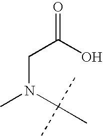 | 403.38 |
| 68 | CH3 | H | 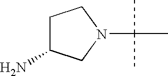 | 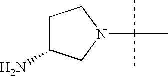 | 397.47 |
| 69 | H | CH3 | 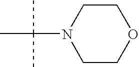 | 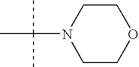 | 399.43 |
| 70 | H | CH3 | 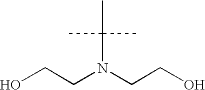 | 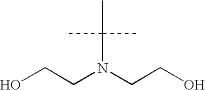 | 435.47 |
| 71 | H | CH3 | 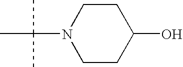 | 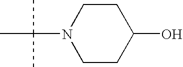 | 427.49 |
| 72 | H | CH3 | 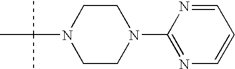 | 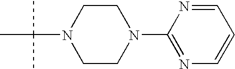 | 553.61 |
| 73 | H | CH3 | 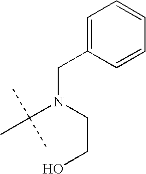 | 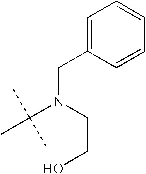 | 527.61 |
| 74 | H | CH3 | 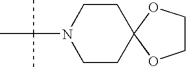 | 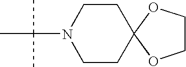 | 511.56 |
| 75 | H | CH3 | 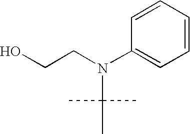 | 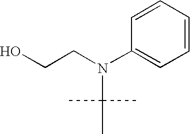 | 499.55 |
| 76 | H | CH3 | 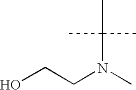 | 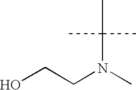 | 375.41 |
| 77 | H | CH3 | 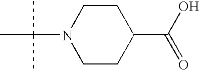 | 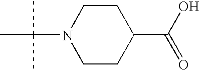 | 483.51 |
| 78 | H | CH3 | 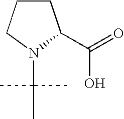 | 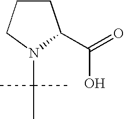 | 455.46 |
| 79 | H | CH3 | 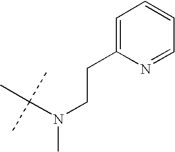 | 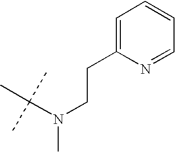 | 497.58 |
| 80 | H | CH3 | 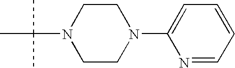 | 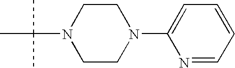 | 551.64 |
| 81 | H | CH3 | 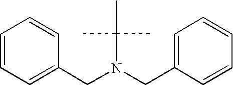 | 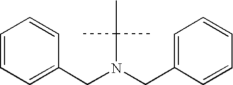 | 619.75 |
| 82 | H | CH3 | 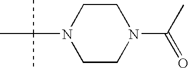 | 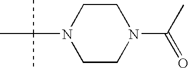 | 481.54 |
| 83 | H | CH3 | 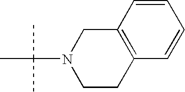 | 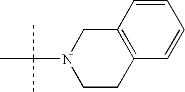 | 491.58 |
| 84 | H | CH3 | 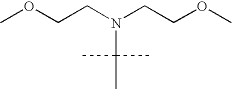 | 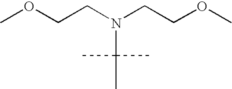 | 491.57 |
| 85 | H | CH3 | 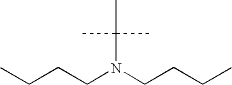 | 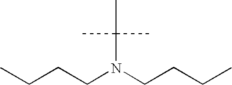 | 483.68 |
| 86 | H | CH3 | 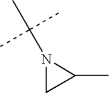 | 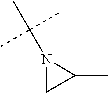 | 339.38 |
| 87 | H | CH3 | 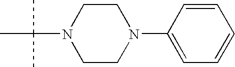 | 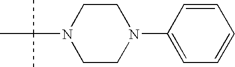 | 549.66 |
| 88 | H | CH3 | 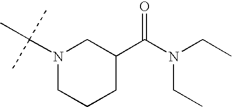 | 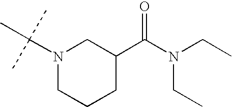 | 593.75 |
| 89 | H | CH3 | 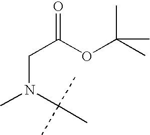 | 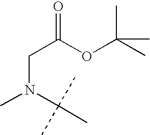 | 515.59 |
| 90 | H | CH3 | 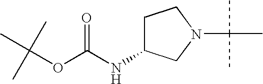 | 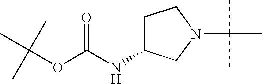 | 597.70 |
| 91 | H | CH3 | 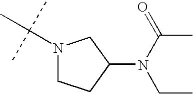 | 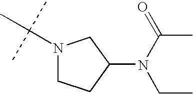 | 537.65 |
| 92 | H | CH3 | 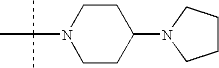 | 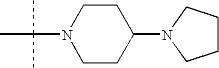 | 533.70 |
| 93 | H | CH3 | 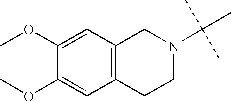 | 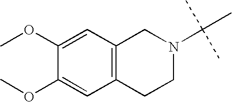 | 611.68 |
| 94 | H | CH3 | 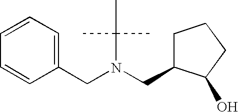 | 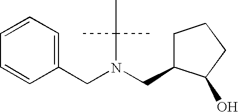 | 635.79 |
| 95 | H | CH3 | 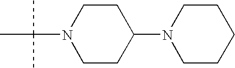 | 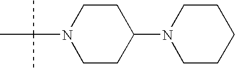 | 561.76 |
| 96 | H | CH3 | 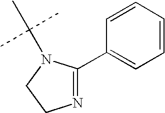 | 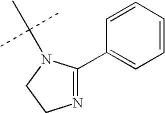 | 517.58 |
| 97 | H | CH3 | 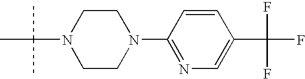 | 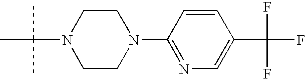 | 687.63 |
| 98 | H | CH3 | 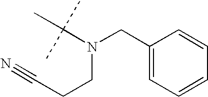 | 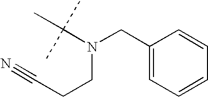 | 545.63 |
| 99 | H | CH3 | 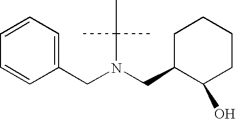 | 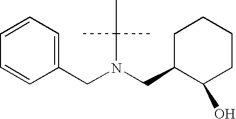 | 663.84 |
| 100 | H | CH3 | 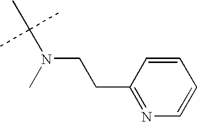 | 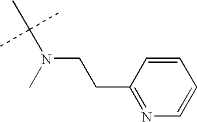 | 497.58 |
| 101 | H | CH3 | 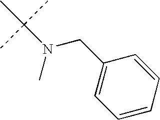 | 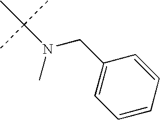 | 467.56 |
| 102 | H | CH3 | 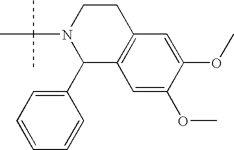 | 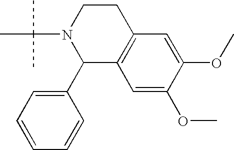 | 763.88 |
| 103 | H | CH3 | 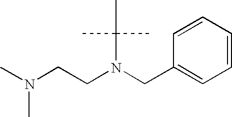 | 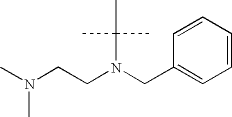 | 581.75 |
| 104 | H | CH3 | 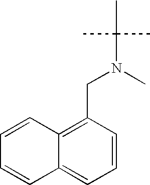 | 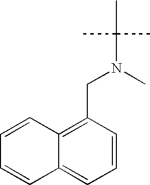 | 567.68 |
| 105 | H | CH3 | 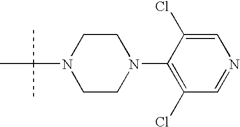 | 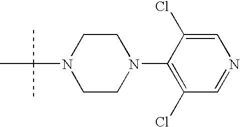 | 689.42 |
| 106 | H | CH3 | 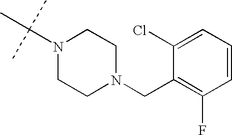 | 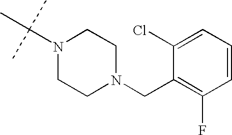 | 682.59 |
| 107 | H | CH3 | 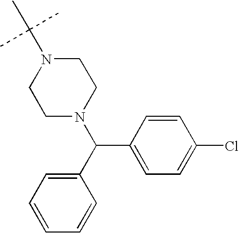 | 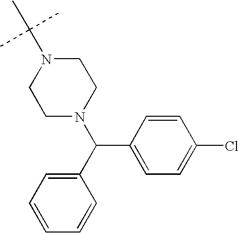 | 798.80 |
| 108 | H | CH3 | 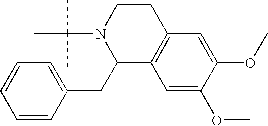 | 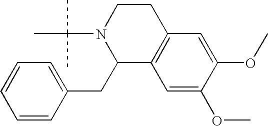 | 791.93 |
| 109 | H | CH3 | 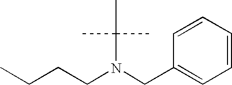 | 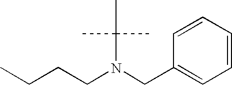 | 551.72 |
| 110 | H | CH3 | 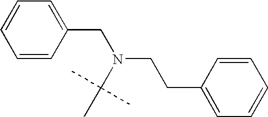 | 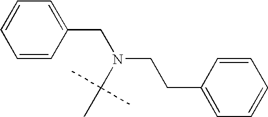 | 647.80 |
| 111 | H | CH3 | 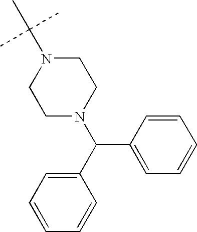 | 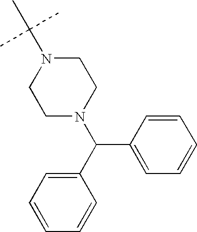 | 729.91 |
| 112 | H | CH3 | 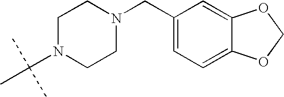 | 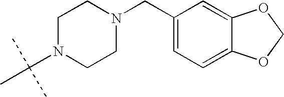 | 665.73 |
| 113 | H | CH3 | 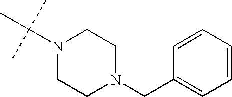 | 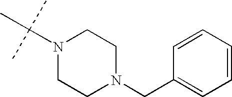 | 577.71 |
| 114 | H | CH3 | 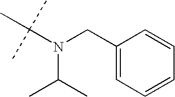 | 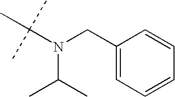 | 523.66 |
| 115 | H | CH3 | 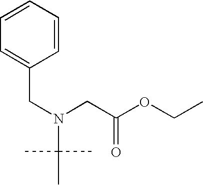 | 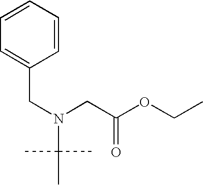 | 611.68 |
| 116 | H | CH3 | 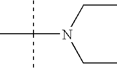 | 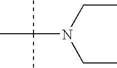 | 371.47 |
| 117 | H | CH3 | 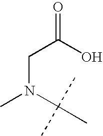 | 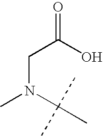 | 403.38 |
| 118 | H | CH3 | 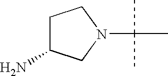 | 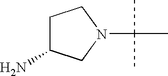 | 397.47 |
| 119 | H | H | 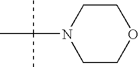 | 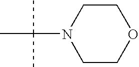 | 385.42 |

TABLE 2

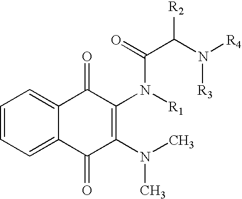

| Cmpd | R₁ | R₂ | 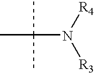 | MW |
|---|---|---|---|---|
| 1 | CH3 | H | 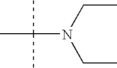 | 343.44 |
| 2 | H | H | 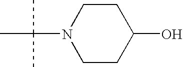 | 357.42 |
| 3 | H | CH3 | 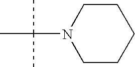 | 355.45 |
| 4 | H | CH3 | 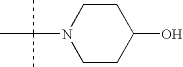 | 371.45 |
| 5 | H | H | 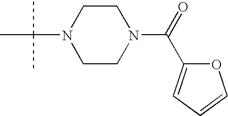 | 436.48 |
| 6 | H | H | 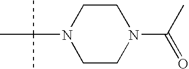 | 384.44 |
| 7 | H | CH3 | 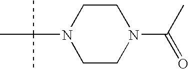 | 398.47 |
| 8 | H | CH3 | 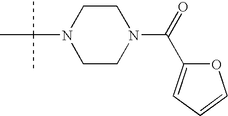 | 450.50 |
| 9 | CH3 | H | 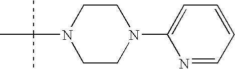 | 433.52 |
| 10 | CH3 | H | 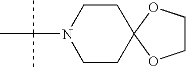 | 413.48 |
| 11 | CH3 | H | 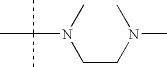 | 372.48 |
| 12 | CH3 | H | 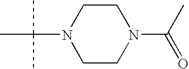 | 398.47 |
| 13 | CH3 | H | 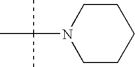 | 355.45 |
| 14 | CH3 | H | 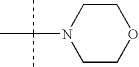 | 357.42 |
| 15 | CH3 | H | 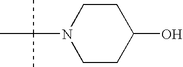 | 371.45 |
| 16 | CH3 | H | 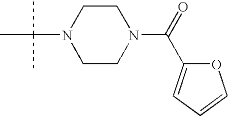 | 450.50 |
| 17 | CH3 | H | 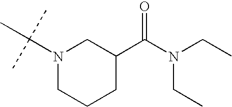 | 454.58 |
| 18 | CH3 | H | 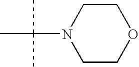 | 357.42 |
| 19 | CH3 | H | 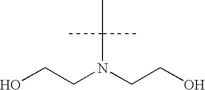 | 375.43 |
| 20 | CH3 | H | 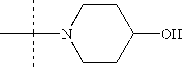 | 371.45 |
| 21 | CH3 | H | 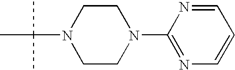 | 434.51 |
| 22 | CH3 | H | 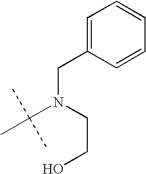 | 421.50 |
| 23 | CH3 | H | 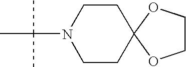 | 413.48 |
| 24 | CH3 | H | 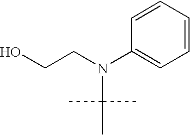 | 407.48 |
| 25 | CH3 | H | 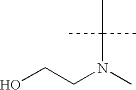 | 345.41 |
| 26 | CH3 | H | 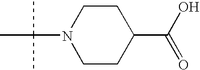 | 399.46 |
| 27 | CH3 | H | 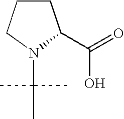 | 385.43 |
| 28 | CH3 | H | 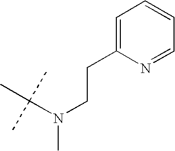 | 406.49 |
| 29 | CH3 | H | 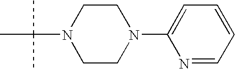 | 433.52 |
| 30 | CH3 | H | 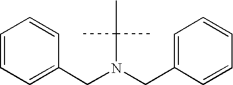 | 467.57 |
| 31 | CH3 | H | 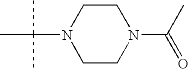 | 398.47 |
| 32 | CH3 | H | 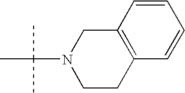 | 403.49 |
| 33 | CH3 | H | 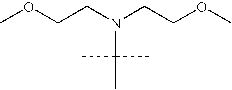 | 403.49 |
| 34 | CH3 | H | 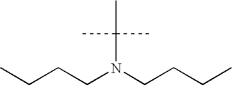 | 399.54 |
| 35 | CH3 | H | 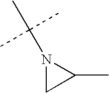 | 327.39 |
| 36 | CH3 | H | 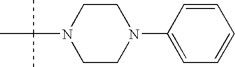 | 432.53 |
| 37 | CH3 | H | 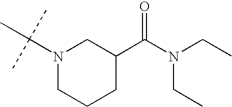 | 454.58 |
| 38 | CH3 | H | 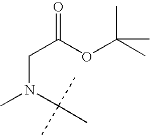 | 415.50 |
| 39 | CH3 | H | 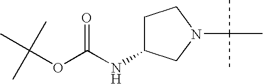 | 456.55 |
| 40 | CH3 | H | 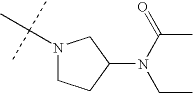 | 426.52 |
| 41 | CH3 | H | 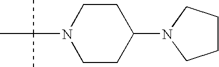 | 424.55 |
| 42 | CH3 | H | 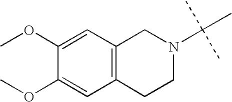 | 463.54 |
| 43 | CH3 | H | 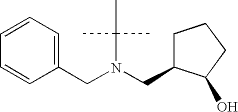 | 475.60 |
| 44 | CH3 | H | 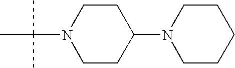 | 438.58 |
| 45 | CH3 | H | 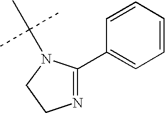 | 416.49 |
| 46 | CH3 | H | 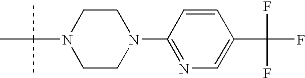 | 501.52 |
| 47 | CH3 | H | 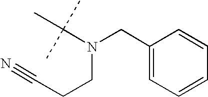 | 430.51 |
| 48 | CH3 | H | 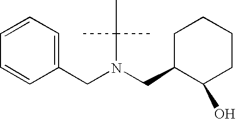 | 489.62 |
| 49 | CH3 | H | 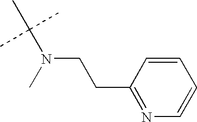 | 406.49 |
| 50 | CH3 | H | 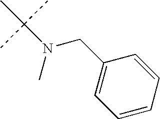 | 391.48 |
| 51 | CH3 | H | 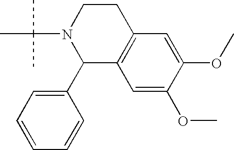 | 539.64 |
| 52 | CH3 | H | | 372.48 |
| 53 | CH3 | H | 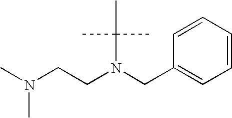 | 448.57 |
| 54 | CH3 | H | 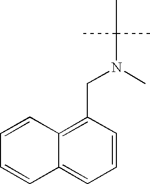 | 441.54 |
| 55 | CH3 | H | 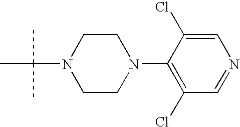 | 502.41 |
| 56 | CH3 | H | 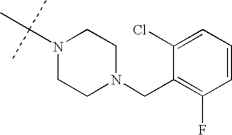 | 498.99 |
| 57 | CH3 | H | 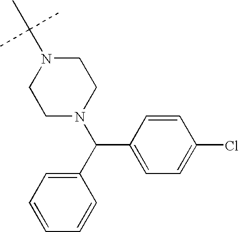 | 557.10 |
| 58 | CH3 | H | 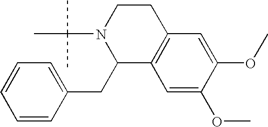 | 553.66 |
| 59 | CH3 | H | 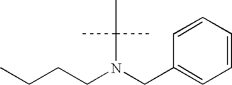 | 433.56 |
| 60 | CH3 | H | 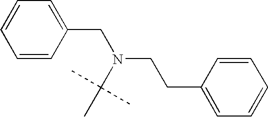 | 481.60 |
| 61 | CH3 | H | 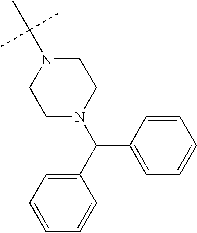 | 522.65 |
| 62 | CH3 | H | 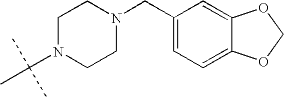 | 490.57 |
| 63 | CH3 | H | 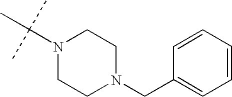 | 446.56 |
| 64 | CH3 | H | 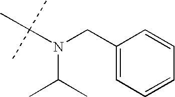 | 419.53 |
| 65 | CH3 | H | 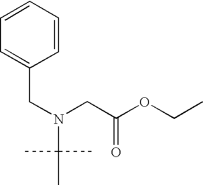 | 463.54 |
| 66 | CH3 | H | 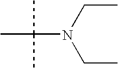 | 315.38 |
| 67 | CH3 | H | 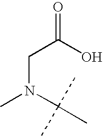 | 359.39 |
| 68 | CH3 | H | 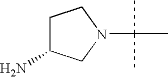 | 356.43 |
| 69 | H | CH3 | 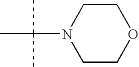 | 357.42 |
| 70 | H | CH3 | 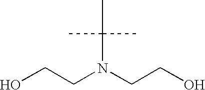 | 375.43 |
| 71 | H | CH3 | 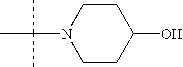 | 371.45 |
| 72 | H | CH3 | 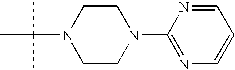 | 434.51 |
| 73 | H | CH3 | 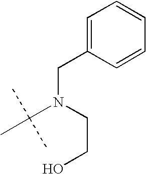 | 421.50 |
| 74 | H | CH3 | 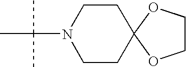 | 413.48 |
| 75 | H | CH3 | 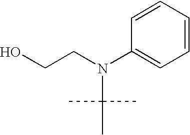 | 407.48 |
| 76 | H | CH3 | 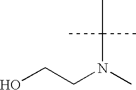 | 345.41 |
| 77 | H | CH3 | 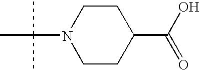 | 399.46 |
| 78 | H | CH3 | 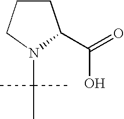 | 385.43 |
| 79 | H | CH3 | 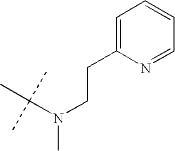 | 406.49 |
| 80 | H | CH3 | 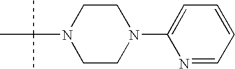 | 433.52 |
| 81 | H | CH3 | 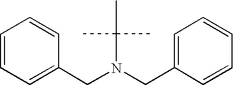 | 467.57 |
| 82 | H | CH3 | 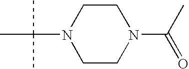 | 398.47 |
| 83 | H | CH3 | 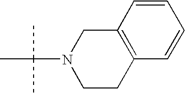 | 403.49 |
| 84 | H | CH3 | 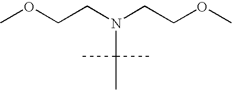 | 403.49 |
| 85 | H | CH3 | 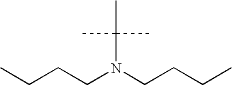 | 399.54 |
| 86 | H | CH3 | 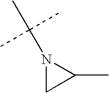 | 327.39 |
| 87 | H | CH3 | 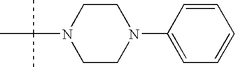 | 432.53 |
| 88 | H | CH3 | 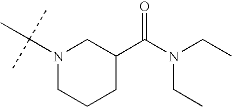 | 454.58 |
| 89 | H | CH3 | 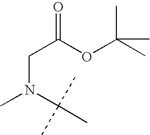 | 415.50 |
| 90 | H | CH3 | 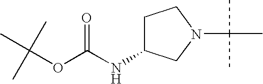 | 456.55 |
| 91 | H | CH3 | 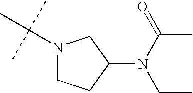 | 426.52 |
| 92 | H | CH3 | | 424.55 |
| 93 | H | CH3 | 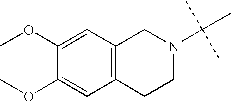 | 463.54 |
| 94 | H | CH3 | 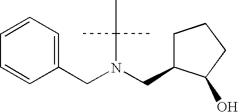 | 475.60 |
| 95 | H | CH3 | 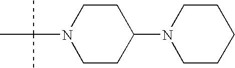 | 438.58 |
| 96 | H | CH3 | 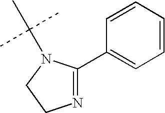 | 416.49 |
| 97 | H | CH3 | 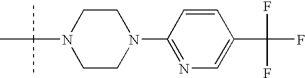 | 501.52 |
| 98 | H | CH3 | 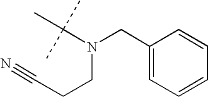 | 430.51 |
| 99 | H | CH3 | 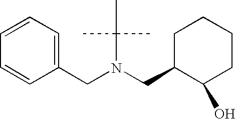 | 489.62 |
| 100 | H | CH3 | 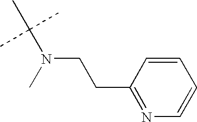 | 406.49 |
| 101 | H | CH3 | 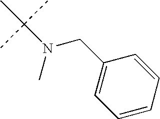 | 391.48 |
| 102 | H | CH3 | 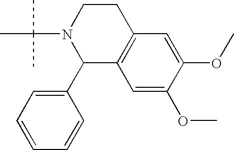 | 539.64 |
| 103 | H | CH3 | 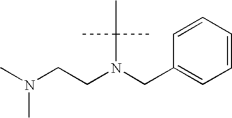 | 448.57 |
| 104 | H | CH3 | 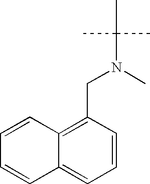 | 441.54 |
| 105 | H | CH3 | 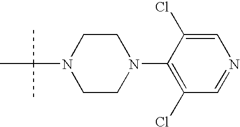 | 502.41 |
| 106 | H | CH3 | 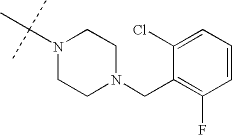 | 498.99 |
| 107 | H | CH3 | 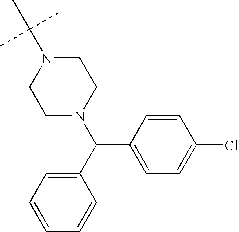 | 557.10 |
| 108 | H | CH3 | 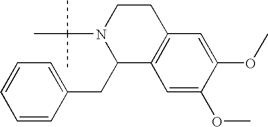 | 553.66 |
| 109 | H | CH3 | 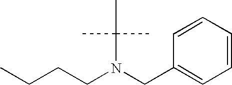 | 433.56 |
| 110 | H | CH3 | 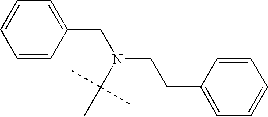 | 481.60 |
| 111 | H | CH3 | 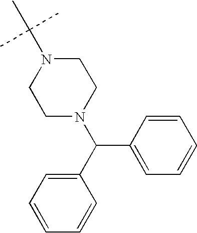 | 522.65 |
| 112 | H | CH3 | 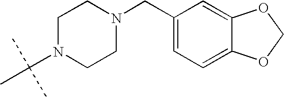 | 490.57 |
| 113 | H | CH3 | 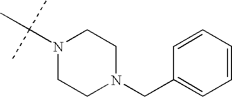 | 446.56 |
| 114 | H | CH3 | 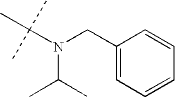 | 419.53 |
| 115 | H | CH3 | 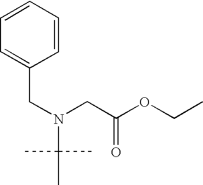 | 463.54 |
| 116 | H | CH3 | 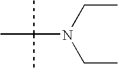 | 343.44 |
| 117 | H | CH3 | 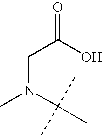 | 359.39 |
| 118 | H | CH3 | 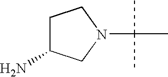 | 356.43 |
| 119 | H | H | 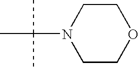 | 343.39 |

TABLE 3

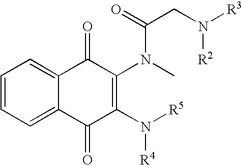

| Cmpd | 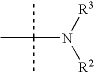 | 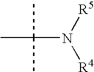 | MW |
|---|---|---|---|
| 1 | 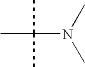 | 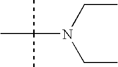 | 343.42 |
| 2 | 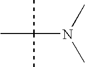 | 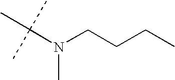 | 357.45 |
| 3 | 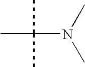 | 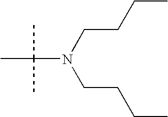 | 399.53 |
| 4 | 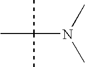 | 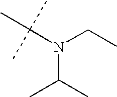 | 357.45 |
| 5 | 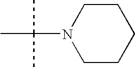 | 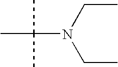 | 383.48 |
| 6 | 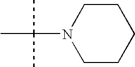 | 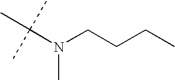 | 397.51 |
| 7 | 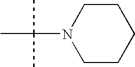 | 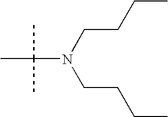 | 439.59 |
| 8 | 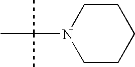 | 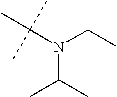 | 397.51 |
| 9 | 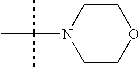 | 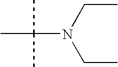 | 385.46 |
| 10 | 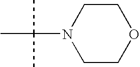 | 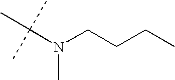 | 399.48 |
| 11 | 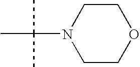 | 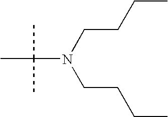 | 441.56 |
| 12 | 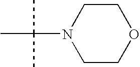 | 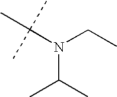 | 399.48 |
| 13 | 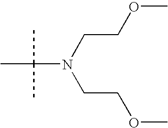 | 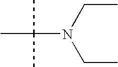 | 431.53 |
| 14 | 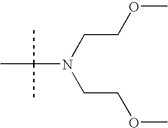 | 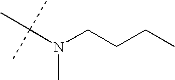 | 445.55 |
| 15 | 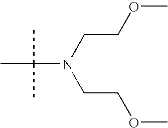 | 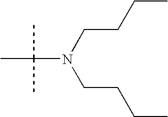 | 487.63 |
| 16 | 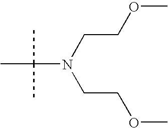 | 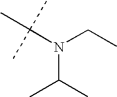 | 445.55 |
| 17 | 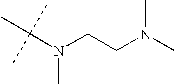 | 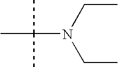 | 400.51 |
| 18 | 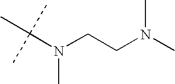 | 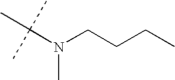 | 414.54 |
| 19 | 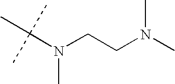 | 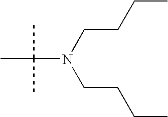 | 456.62 |
| 20 | 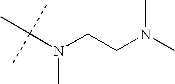 | 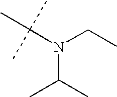 | 414.54 |
| 21 | 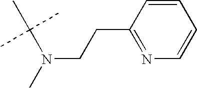 | 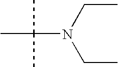 | 434.53 |
| 22 | 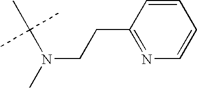 | 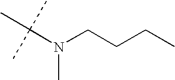 | 448.56 |
| 23 | 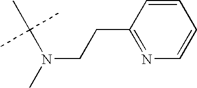 | 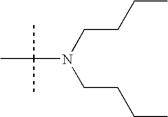 | 490.64 |
| 24 | 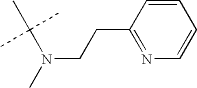 | 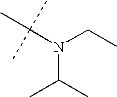 | 448.56 |

TABLE 4

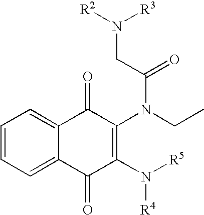

| Cmpd | 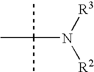 | 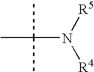 | MW |
|---|---|---|---|
| 1 | 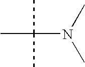 | 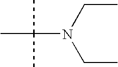 | 357.45 |
| 2 | 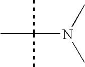 | 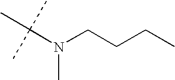 | 371.47 |
| 3 | 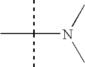 | 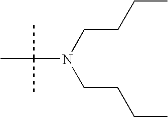 | 413.55 |
| 4 | 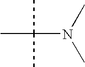 | 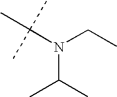 | 371.47 |
| 5 | 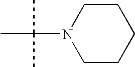 | 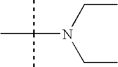 | 397.51 |
| 6 | 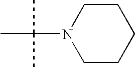 | 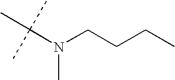 | 411.54 |
| 7 | 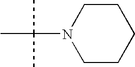 | 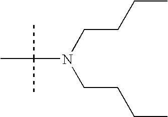 | 453.62 |
| 8 | 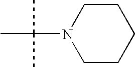 | 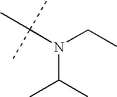 | 411.54 |
| 9 | 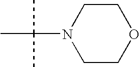 | 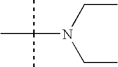 | 399.48 |
| 10 | 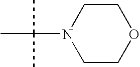 | 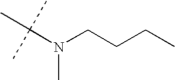 | 413.51 |
| 11 | 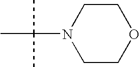 | 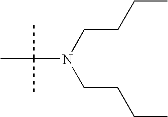 | 455.59 |
| 12 | 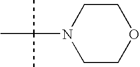 | 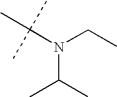 | 413.51 |
| 13 | 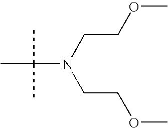 | 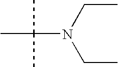 | 445.55 |
| 14 | 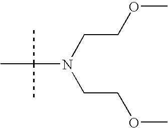 | 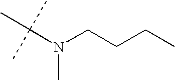 | 459.58 |
| 15 | 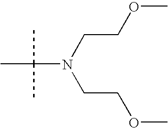 | 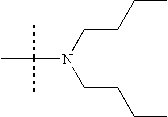 | 501.66 |
| 16 | 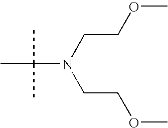 | 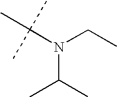 | ethylamine |
| 17 | 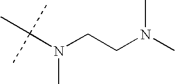 | 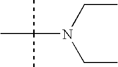 | 414.54 |
| 18 | 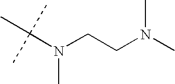 | 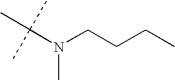 | 428.57 |
| 19 | 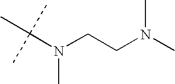 | 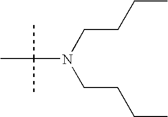 | 470.65 |
| 20 | 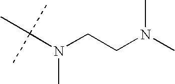 | 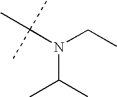 | 428.57 |
| 21 | 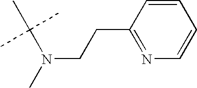 | 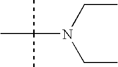 | 448.56 |
| 22 | 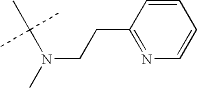 | 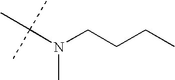 | 462.58 |
| 23 | 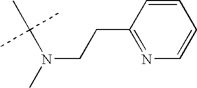 | 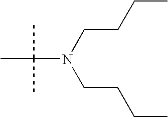 | 504.66 |
| 24 | 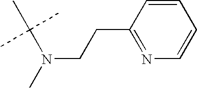 | 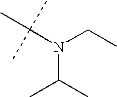 | 462.58 |

TABLE 5

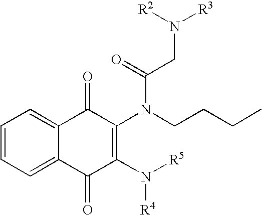

| Cpmd | 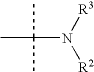 | 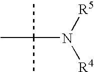 | MW |
|---|---|---|---|
| 1 | 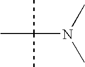 | 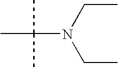 | 385.50 |
| 2 | 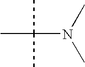 | 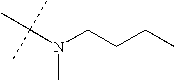 | 399.53 |
| 3 | 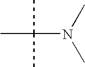 | 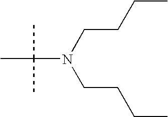 | 441.61 |
| 4 | 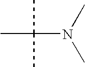 | 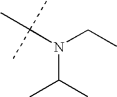 | 399.53 |
| 5 | 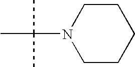 | 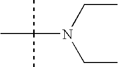 | 425.56 |
| 6 | 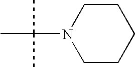 | 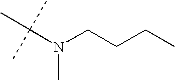 | 439.59 |
| 7 | 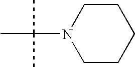 | 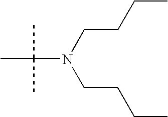 | 481.67 |
| 8 | 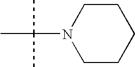 | 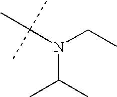 | 439.59 |
| 9 | 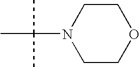 | 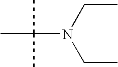 | 427.54 |
| 10 | 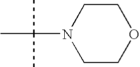 | 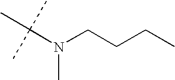 | 441.56 |
| 11 | 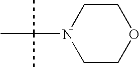 | 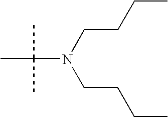 | 483.64 |
| 12 | 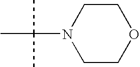 | 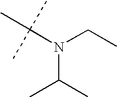 | 441.56 |
| 13 | 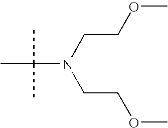 | 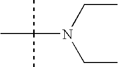 | 473.6 |
| 14 | 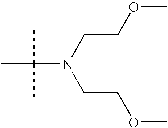 | 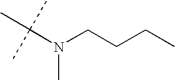 | 487.63 |
| 15 | 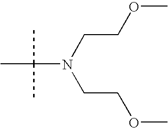 | 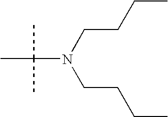 | 529.71 |
| 16 | 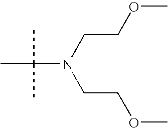 | 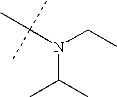 | 487.63 |
| 17 | 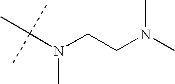 | 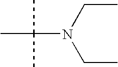 | 442.59 |
| 18 | 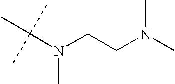 | 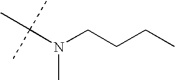 | 456.62 |
| 19 | 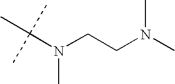 | 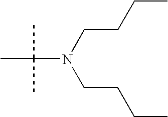 | 498.7 |
| 20 | 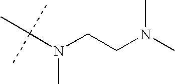 | 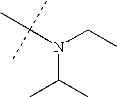 | 456.62 |
| 21 | 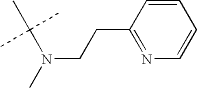 | 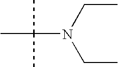 | 476.61 |
| 22 | 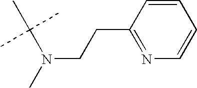 | 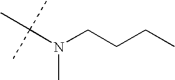 | 490.64 |
| 23 | 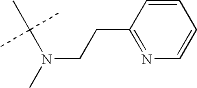 | 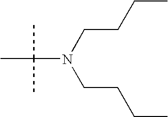 | 532.72 |
| 24 | 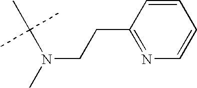 | 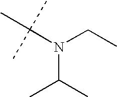 | 490.64 |

TABLE 6

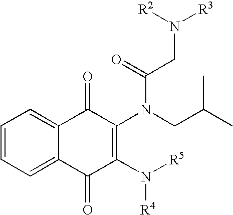

| Cmpd | 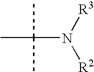 | 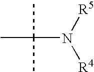 | MW |
|---|---|---|---|
| 1 | 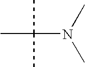 | 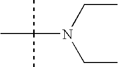 | 385.50 |
| 2 | 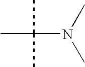 | 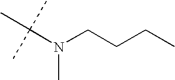 | 399.53 |
| 3 | 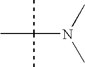 | 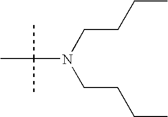 | 441.61 |
| 4 | 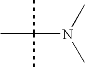 | 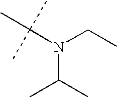 | 399.53 |
| 5 | 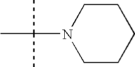 | 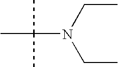 | 425.56 |
| 6 | 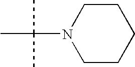 | 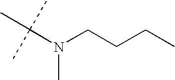 | 439.59 |
| 7 | 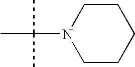 | 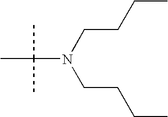 | 481.67 |
| 8 | 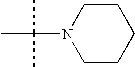 | 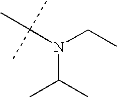 | 439.59 |
| 9 | 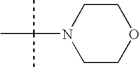 | 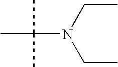 | 427.54 |
| 10 | 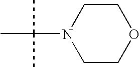 | 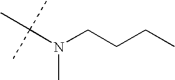 | 441.56 |
| 11 | 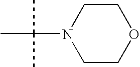 | 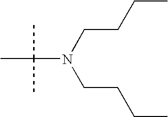 | 483.64 |
| 12 | 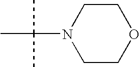 | 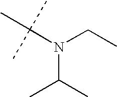 | 441.56 |
| 13 | 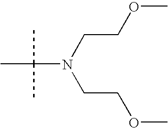 | 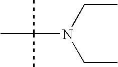 | 473.6 |
| 14 | 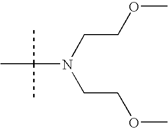 | 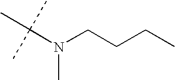 | 487.63 |
| 15 | 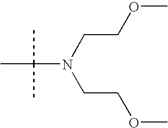 | 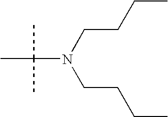 | 529.71 |
| 16 | 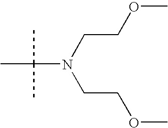 | 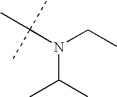 | 487.63 |
| 17 | 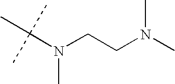 | 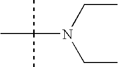 | 442.59 |
| 18 | 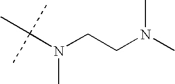 | 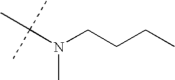 | 456.62 |
| 19 | 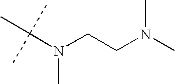 | 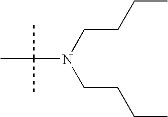 | 498.7 |
| 20 | 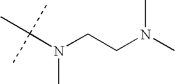 | 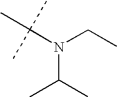 | 456.62 |
| 21 | 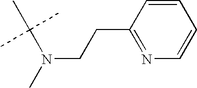 | 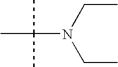 | 476.61 |
| 22 | 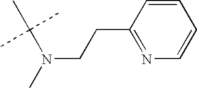 | 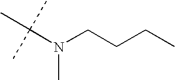 | 490.64 |
| 23 | 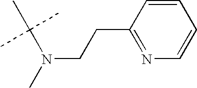 | 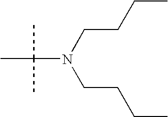 | 532.72 |
| 24 | 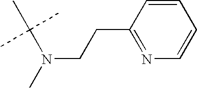 | 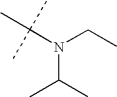 | 490.64 |

TABLE 7

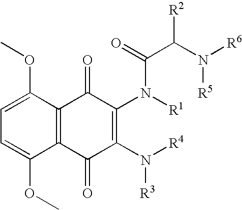

| Cmpd | R₁ | R₂ | 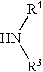 | 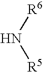 | MW |
|---|---|---|---|---|---|
| 1 | CH3 | H | 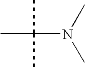 | 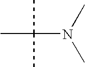 | 375.42 |
| 2 | CH3 | H | 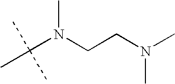 | 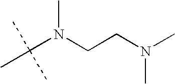 | 489.61 |
| 3 | CH3 | H | 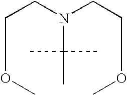 | 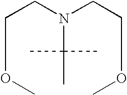 | 551.63 |
| 4 | CH3 | H | 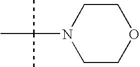 | 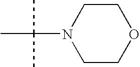 | 459.49 |
| 5 | CH3 | H | 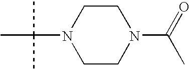 | 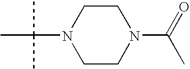 | 541.60 |
| 6 | H | CH3 | 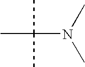 | 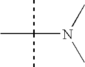 | 375.42 |
| 7 | H | CH3 | 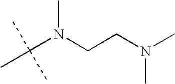 | 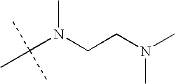 | 489.61 |
| 8 | H | CH3 | 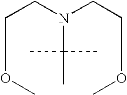 | 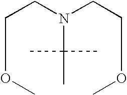 | 551.63 |
| 9 | H | CH3 | 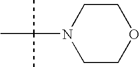 | 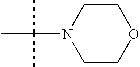 | 459.49 |
| 10 | H | CH3 | 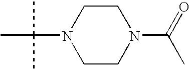 | 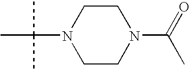 | 541.60 |

TABLE 8

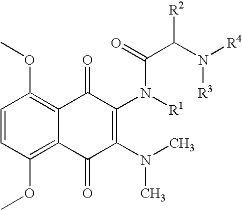

| Cmpd | R₁ | R₂ | 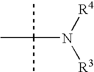 | MW |
|---|---|---|---|---|
| 1 | CH3 | H | 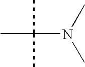 | 375.42 |
| 2 | CH3 | H | 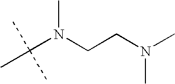 | 432.51 |
| 3 | CH3 | H | 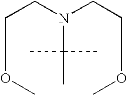 | 463.52 |
| 4 | CH3 | H | 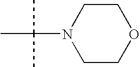 | 417.45 |
| 5 | CH3 | H | 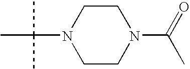 | 458.51 |
| 6 | H | CH3 | 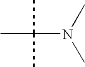 | 375.42 |
| 7 | H | CH3 | 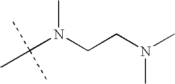 | 432.51 |
| 8 | H | CH3 | 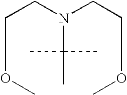 | 463.52 |
| 9 | H | CH3 | 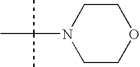 | 417.45 |
| 10 | H | CH3 | 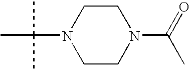 | 458.51 |

TABLE 9

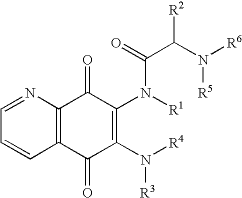

| Cmpd | R₁ | R₂ | 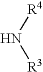 | 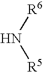 | MW |
|---|---|---|---|---|---|
| 1 | CH3 | H | 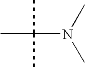 | 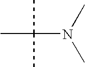 | 316.35 |
| 2 | CH3 | H | 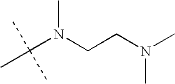 | 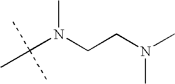 | 430.54 |
| 3 | CH3 | H | 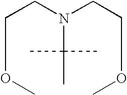 | 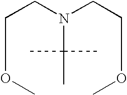 | 492.57 |
| 4 | CH3 | H | 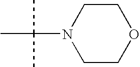 | 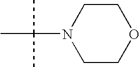 | 400.43 |
| 5 | CH3 | H | 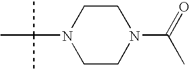 | 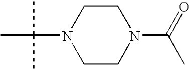 | 482.53 |
| 6 | H | CH3 | 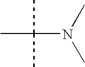 | 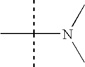 | 316.35 |
| 7 | H | CH3 | 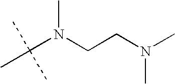 | 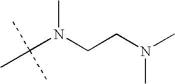 | 430.54 |
| 8 | H | CH3 | 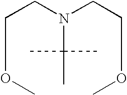 | 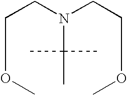 | 492.57 |
| 9 | H | CH3 | 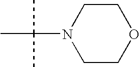 | 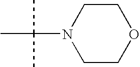 | 400.43 |
| 10 | H | CH3 | 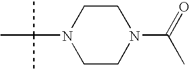 | 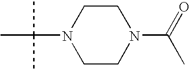 | 482.53 |

TABLE 10

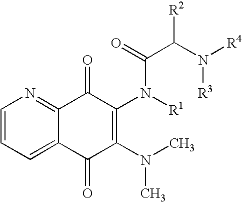

| Cmpd | R₁ | R₂ | 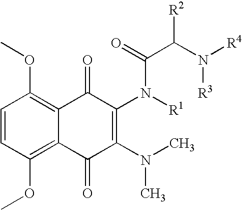 | MW |
|---|---|---|---|---|
| 1 | CH3 | H | 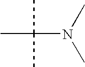 | 316.35 |
| 2 | CH3 | H | 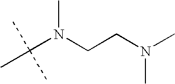 | 373.45 |
| 3 | CH3 | H | 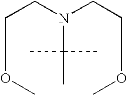 | 404.46 |
| 4 | CH3 | H | 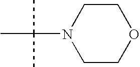 | 358.39 |
| 5 | CH3 | H | 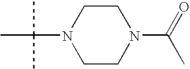 | 399.44 |
| 6 | H | CH3 | 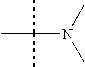 | 316.35 |
| 7 | H | CH3 | 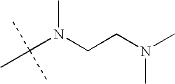 | 373.45 |
| 8 | H | CH3 | 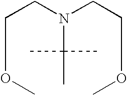 | 404.46 |
| 9 | H | CH3 | 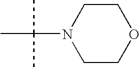 | 358.39 |
| 10 | H | CH3 | 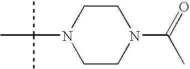 | 399.44 |

TABLE 11

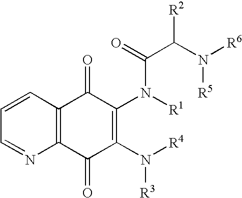

| Cmpd | R₁ | R₂ | 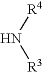 | 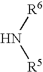 | MW |
|---|---|---|---|---|---|
| 1 | CH3 | H | 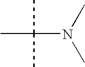 | 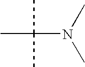 | 316.35 |
| 2 | CH3 | H | 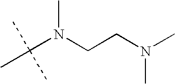 | 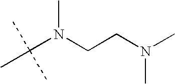 | 430.54 |
| 3 | CH3 | H | 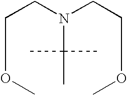 | 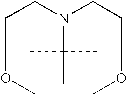 | 492.57 |
| 4 | CH3 | H | 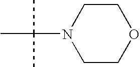 | 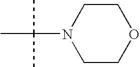 | 400.43 |
| 5 | CH3 | H | 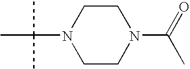 | 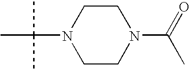 | 482.53 |
| 6 | H | CH3 | 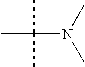 | 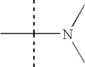 | 316.35 |
| 7 | H | CH3 | 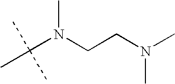 | 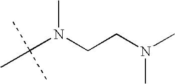 | 430.54 |
| 8 | H | CH3 | 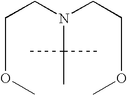 | 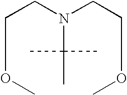 | 492.57 |
| 9 | H | CH3 | 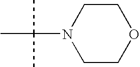 | 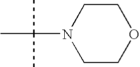 | 400.43 |
| 10 | H | CH3 | 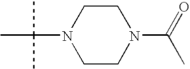 | 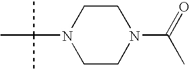 | 482.53 |

TABLE 12

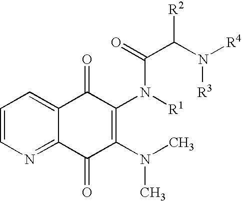

| Cmpd | R₁ | R₂ | 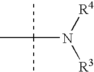 | MW |
|---|---|---|---|---|
| 1 | CH3 | H | 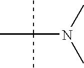 | 316.35 |
| 2 | CH3 | H | 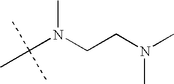 | 373.45 |
| 3 | CH3 | H | 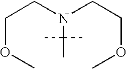 | 404.46 |
| 4 | CH3 | H | 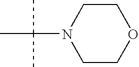 | 358.39 |
| 5 | CH3 | H | 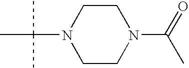 | 399.44 |
| 6 | H | CH3 | 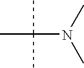 | 316.35 |
| 7 | H | CH3 | 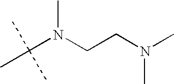 | 373.45 |
| 8 | H | CH3 | 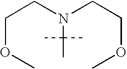 | 404.46 |
| 9 | H | CH3 | 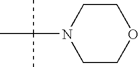 | 358.39 |
| 10 | H | CH3 | 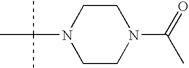 | 399.44 |

TABLE 13

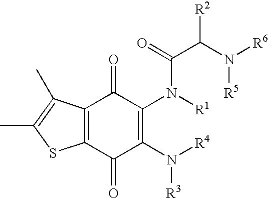

| Cmpd | R₁ | R₂ | 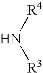 | 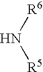 | MW |
|---|---|---|---|---|---|
| 1 | CH3 | H | 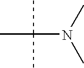 | 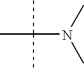 | 349.45 |
| 2 | CH3 | H | 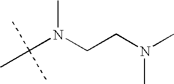 | 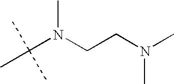 | 463.64 |
| 3 | CH3 | H | 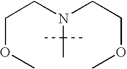 | 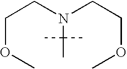 | 525.66 |
| 4 | CH3 | H | 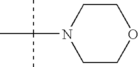 | 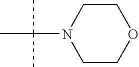 | 433.52 |
| 5 | CH3 | H | 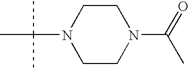 | 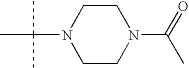 | 515.63 |
| 6 | H | CH3 | 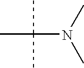 | 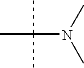 | 349.45 |
| 7 | H | CH3 | 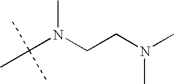 | 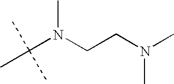 | 463.64 |
| 8 | H | CH3 | 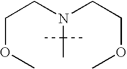 | 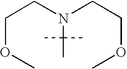 | 525.66 |
| 9 | H | CH3 | 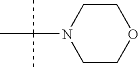 | 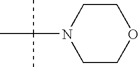 | 433.52 |
| 10 | H | CH3 | 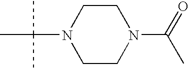 | 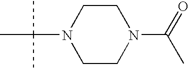 | 515.63 |

TABLE 14

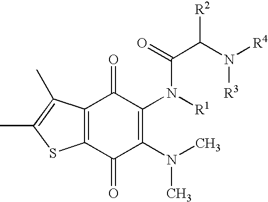

| Cmpd | R₁ | R₂ | 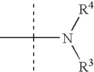 | MW |
|---|---|---|---|---|
| 1 | CH3 | H | 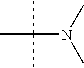 | 349.45 |
| 2 | CH3 | H | 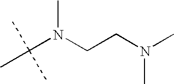 | 406.54 |
| 3 | CH3 | H | 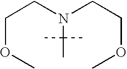 | 437.55 |
| 4 | CH3 | H | 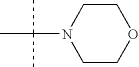 | 391.48 |
| 5 | CH3 | H | 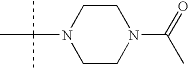 | 432.54 |
| 6 | H | CH3 | 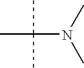 | 349.45 |
| 7 | H | CH3 | 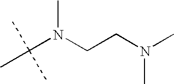 | 406.54 |
| 8 | H | CH3 | 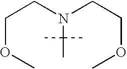 | 437.55 |
| 9 | H | CH3 | 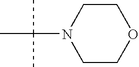 | 391.48 |
| 10 | H | CH3 | 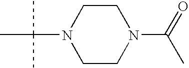 | 432.54 |

TABLE 15

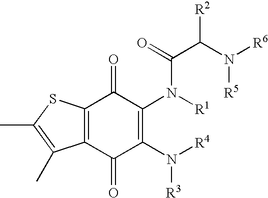

| Cmpd | R₁ | R₂ | 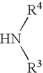 | 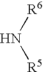 | MW |
|---|---|---|---|---|---|
| 1 | CH3 | H | 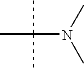 | 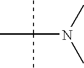 | 349.45 |
| 2 | CH3 | H | 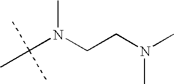 | 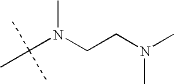 | 463.64 |
| 3 | CH3 | H | 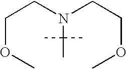 | 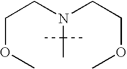 | 525.66 |
| 4 | CH3 | H | 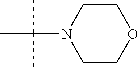 | 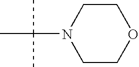 | 433.52 |
| 5 | CH3 | H | 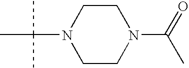 | 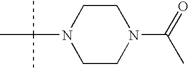 | 515.63 |
| 6 | H | CH3 | 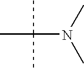 | 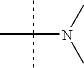 | 349.45 |
| 7 | H | CH3 | 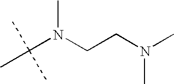 | 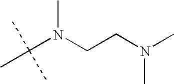 | 463.64 |
| 8 | H | CH3 | 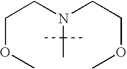 | 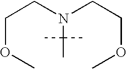 | 525.66 |
| 9 | H | CH3 | 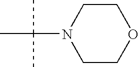 | 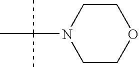 | 433.52 |
| 10 | H | CH3 | 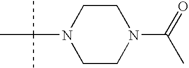 | 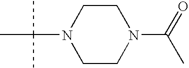 | 515.63 |

TABLE 16

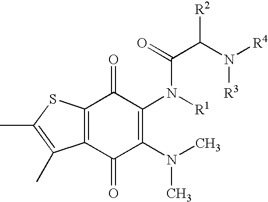

| Cmpd | R₁ | R₂ | 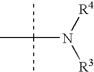 | MW |
|---|---|---|---|---|
| 1 | CH3 | H | 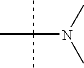 | 349.45 |
| 2 | CH3 | H | 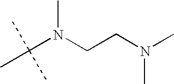 | 406.54 |
| 3 | CH3 | H | 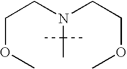 | 437.55 |
| 4 | CH3 | H | 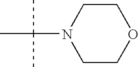 | 391.48 |
| 5 | CH3 | H | 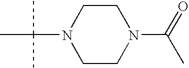 | 432.54 |
| 6 | H | CH3 | 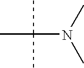 | 349.45 |
| 7 | H | CH3 | 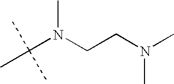 | 406.54 |
| 8 | H | CH3 | 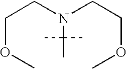 | 437.55 |
| 9 | H | CH3 | 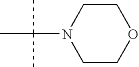 | 391.48 |
| 10 | H | CH3 | 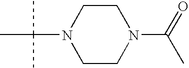 | 432.54 |

TABLE 17

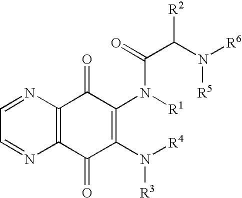

| Cmpd | R₁ | R₂ | 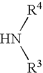 | 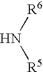 | MW |
|---|---|---|---|---|---|
| 1 | CH3 | H | 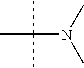 | 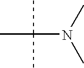 | 317.34 |
| 2 | CH3 | H | 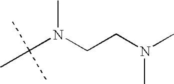 | 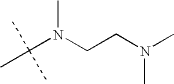 | 431.53 |
| 3 | CH3 | H | 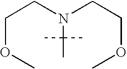 | 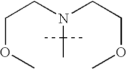 | 493.55 |
| 4 | CH3 | H | 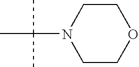 | 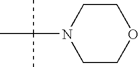 | 401.42 |
| 5 | CH3 | H | 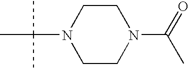 | 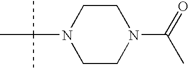 | 483.52 |
| 6 | H | CH3 | 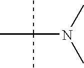 | 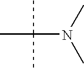 | 317.34 |
| 7 | H | CH3 | 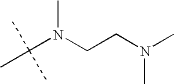 | 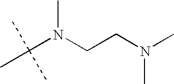 | 431.53 |
| 8 | H | CH3 | 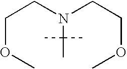 | 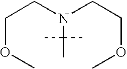 | 493.55 |
| 9 | H | CH3 | 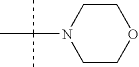 | 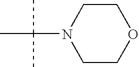 | 401.42 |
| 10 | H | CH3 | 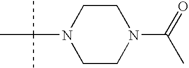 | 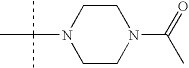 | 483.52 |

TABLE 18

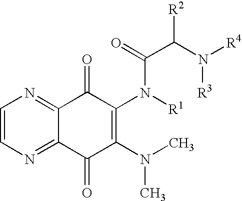

| Cmpd | R₁ | R₂ | 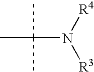 | MW |
|---|---|---|---|---|
| 1 | CH3 | H | 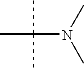 | 317.34 |
| 2 | CH3 | H | 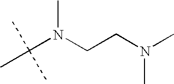 | 374.44 |
| 3 | CH3 | H | 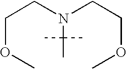 | 405.45 |
| 4 | CH3 | H | 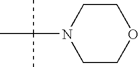 | 359.38 |
| 5 | CH3 | H | 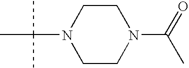 | 400.43 |
| 6 | H | CH3 | 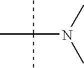 | 317.34 |
| 7 | H | CH3 | 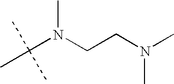 | 374.44 |
| 8 | H | CH3 | 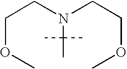 | 405.45 |
| 9 | H | CH3 | 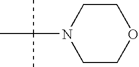 | 359.38 |
| 10 | H | CH3 | 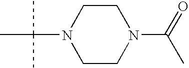 | 400.43 |

TABLE 19

| Gene | Gene | |
|---|---|---|
| No. | Identifier | Gene Name |
| 1 | XM_011929 | RTP801 |
| 2 | NM_004864 | prostate differentiation factor |
| 3 | NM_001657 | amphiregulin (schwannoma-derived growth factor) |
| 4 | XM_033762 | GRB10 |
| 5 | NM_004083 | DNA-damage-inducible transcript 3 |
| 6 | XM_009097 | PPP1R15A |
| 7 | NM_005542 | insulin induced gene 1 |
| 8 | XM_032884 | MGC11324 |
| 9 | XM_052673 | VEGF |
| 10 | NM_007235 | exportin, tRNA (nuclear export receptor for tRNAs) |
| 11 | NM_000179 | mutS homolog 6 (E. coli) |
| 12 | NM_005194 | CCAAT/enhancer binding protein (C/EBP), beta |
| 13 | XM_043412 | CDKN1A |
| 14 | NM_004448 | v-erb-b2 erythroblastic leukemia viral oncogene homolog 2, neuro/glioblastoma de |
| 15 | NM_004526 | MCM2 minichromosome maintenance deficient 2, mitotin (S. cerevisiae) |
| 16 | XM_035627 | UHRF1 |
| 17 | L24498 | GADD45A |
| 18 | NM_005915 | MCM6 minichromosome maintenance deficient 6 (MIS5 homolog, S. pombe) (S. cerevis |
| 19 | NM_004642 | CDK2-associated protein 1 |
| 20 | NM_004629 | Fanconi anemia, complementation group G |
| 21 | NM_022119 | protease, serine, 22 |
| 22 | XM_002003 | STMN1 |
| 23 | NM_014736 | KIAA0101 gene product |
| 24 | NM_002691 | polymerase (DNA directed), delta 1, catalytic subunit 125 kDa |
| 25 | XM_034901 | MSH2 |
| 26 | XM_001284 | MDM4 |
| 27 | XM_018276 | FLJ13782 |
| 28 | NM_004707 | APG12 autophagy 12-like (S. cerevisiae) |
| 29 | NM_004836 | eukaryotic translation initiation factor 2-alpha kinase 3 |
| 30 | XM_008618 | CBX4 |
| 31 | NM_003504 | CDC45 cell division cycle 45-like (S. cerevisiae) |
| 32 | XM_002242 | HAT1 |
| 33 | NM_014331 | solute carrier family 7, (cationic amino acid transporter, y + system) member 11 |
| 34 | NM_003467 | chemokine (C-X-C motif) receptor 4 |
| 35 | XM_002899 | CDC25A |
| 36 | NM_006349 | putative cyclin G1 interacting protein |
| 37 | XM_056035 | PCNA |
| 38 | XM_003511 | EREG |
| 39 | XM_031515 | RAD51 |
| 40 | XM_017925 | EIF4E |
| 41 | NM_001799 | cyclin-dependent kinase 7 (MO15 homolog, Xenopus laevis, cdk-activating kinase) |
| 42 | NM_004990 | methionine-tRNA synthetase |
| 43 | NM_057749 | cyclin E2 |
| 44 | NM_001540 | heat shock 27 kDa protein 1 |
| 45 | NM_005882 | macrophage erythroblast attacher |
| 46 | XM_047059 | SUV39H1 |
| 47 | NM_006156 | neural precursor cell expressed, developmentally down-regulated 8 |
| 48 | NM_016395 | butyrate-induced transcript 1 |
| 49 | XM_012472 | NPIP |
| 50 | NM_018518 | MCM10 minichromosome maintenance deficient 10 (S. cerevisiae) |
| 51 | NM_000194 | hypoxanthine phosphoribosyltransferase 1 (Lesch-Nyhan syndrome) |
| 52 | NM_002359 | v-maf musculoaponeurotic fibrosarcoma oncogene homolog G (avian) |
| 53 | XM_001589 | DVL1 |
| 54 | NM_003276 | thymopoietin |
| 55 | XM_040103 | DLC1 |
| 56 | XM_010272 | RBBP7 |
| 57 | NM_001226 | caspase 6, apoptosis-related cysteine protease |
| 58 | NM_013376 | CDK4-binding protein p34SEI1 |
| 59 | NM_001196 | BH3 interacting domain death agonist |
| 60 | AF317391 | BCL-6 interacting corepressor |
| 61 | NM_002435 | mannose phosphate isomerase |
| 62 | NM_003503 | CDC7 cell division cycle 7-like 1 (S. cerevisiae) |
| 63 | NM_001168 | baculoviral IAP repeat-containing 5 (survivin) |
| 64 | XM_036462 | ACLY |
| 65 | XM_009643 | RBL1 |
| 66 | NM_001424 | epithelial membrane protein 2 |
| 67 | AK057120 | high-mobility group box 1 |
| 68 | XM_051677 | CDKN3 |
| 69 | NM_001379 | DNA (cytosine-5-)-methyltransferase 1 |
| 70 | XM_001668 | PDZK1 |
| 71 | NM_001967 | eukaryotic translation initiation factor 4A, isoform 2 |
| 72 | XM_050297 | XRCC3 |
| 73 | NM_004428 | ephrin-A1 |
| 74 | AB037790 | heme-regulated initiation factor 2-alpha kinase |
| 75 | NM_007306 | breast cancer 1, early onset |
| 76 | NM_004336 | BUB1 budding uninhibited by benzimidazoles 1 homolog (yeast) |
| 77 | NM_031844 | heterogeneous nuclear ribonucleoprotein U (scaffold attachment factor A) |
| 78 | XM_002943 | POLQ |
| 79 | D21262 | nucleolar and coiled-body phosphoprotein 1 |
| 80 | XM_056165 | YWHAH |
| 81 | NM_006609 | mitogen-activated protein kinase kinase kinase 2 |
| 82 | NM_013258 | apoptosis-associated speck-like protein containing a CARD |
| 83 | NM_024602 | hypothetical protein FLJ21156 |
| 84 | NM_005080 | X-box binding protein 1 |
| 85 | NM_004050 | BCL2-like 2 |
| 86 | NM_014454 | p53 regulated PA26 nuclear protein |
| 87 | W28438 | chromosome 14 open reading frame 78 |
| 88 | XM_008802 | RBBP8 |
| 89 | XM_053627 | FGF4 |
| 90 | NM_006727 | cadherin 10, type 2 (T2-cadherin) |
| 91 | NM_005980 | S100 calcium binding protein P |
| 92 | XM_050665 | FH |
| 93 | NM_000432 | myosin, light polypeptide 2, regulatory, cardiac, slow |
| 94 | D16815 | nuclear receptor subfamily 1, group D, member 2 |
| 95 | XM_044825 | SUPT3H |
| 96 | NM_058179 | phosphoserine aminotransferase |
| 97 | XM_018112 | RBBP4 |
| 98 | NM_020386 | HRAS-like suppressor |
| 99 | AK057758 | insulin receptor substrate 3-like |
| 100 | XM_044111 | RIT1 |
| 101 | NM_004313 | arrestin, beta 2 |
| 102 | L26584 | Ras protein-specific guanine nucleotide-releasing factor 1 |
| 103 | NM_005414 | SKI-like |
| 104 | XM_031603 | BUB1B |
| 105 | XM_015963 | SDFR1 |
| 106 | NM_002415 | macrophage migration inhibitory factor (glycosylation-inhibiting factor) |
| 107 | NM_078487 | cyclin-dependent kinase inhibitor 2B (p15, inhibits CDK4) |
| 108 | XM_047707 | KIAA1265 |
| 109 | NM_001065 | tumor necrosis factor receptor superfamily, member 1A |
| 110 | XM_045104 | LGALS3BP |
| 111 | AI053741 | Homo sapiens, clone IMAGE: 4826963, mRNA |
| 112 | NM_003600 | serine/threonine kinase 6 |
| 113 | NM_012112 | chromosome 20 open reading frame 1 |
| 114 | NM_000387 | solute carrier family 25 (carnitine/acylcarnitine translocase), member 20 |
| 115 | NM_005587 | MADS box transcription enhancer factor 2, polypeptide A (myocyte enhancer factor |
| 116 | NM_001892 | casein kinase 1, alpha 1 |
| 117 | NM_016277 | RAB23, member RAS oncogene family |
| 118 | NM_003094 | small nuclear ribonucleoprotein polypeptide E |
| 119 | NM_006623 | phosphoglycerate dehydrogenase |
| 120 | NM_005441 | chromatin assembly factor 1, subunit B (p60) |
| 121 | NM_002659 | plasminogen activator, urokinase receptor |
| 122 | NM_000057 | Bloom syndrome |
| 123 | NM_001202 | bone morphogenetic protein 4 |
| 124 | NM_003289 | tropomyosin 2 (beta) |
| 125 | XM_003325 | CCNA2 |
| 126 | XM_032813 | HUMGT198A |
| 127 | NM_006403 | enhancer of filamentation 1 |
| 128 | NM_006289 | talin 1 |
| 129 | NM_003405 | tyrosine 3-monooxygenase/tryptophan 5-monooxygenase activation protein, eta poly |
| 130 | NM_000368 | tuberous sclerosis 1 |
| 131 | BC008826 | PAX3 |
| 132 | NM_003908 | eukaryotic translation initiation factor 2, subunit 2 beta, 38 kDa |
| 133 | NM_004282 | BCL2-associated athanogene 2 |
| 134 | XM_010777 | ICAP-1A |
| 135 | XM_034350 | ANXA3 |
| 136 | NM_004965 | high-mobility group nucleosome binding domain 1 |
| 137 | NM_001216 | carbonic anhydrase IX |
| 138 | NM_006325 | RAN, member RAS oncogene family |
| 139 | NM_006516 | solute carrier family 2 (facilitated glucose transporter), member 1 |
| 140 | NM_003657 | breast carcinoma amplified sequence 1 |
| 141 | NM_004417 | dual specificity phosphatase 1 |
| 142 | M94362 | LMNB2 |
| 143 | XM_057994 | SDHA |
| 144 | XM_043451 | PIM1 |
| 145 | NM_021005 | nuclear receptor subfamily 2, group F, member 2 |
| 146 | XM_049928 | CARD14 |
| 147 | AA017553 | ESTs |
| 148 | NM_004905 | antioxidant protein 2 |
| 149 | NM_001274 | CHK1 checkpoint homolog (S. pombe) |
| 150 | NM_002483 | carcinoembryonic antigen-related cell adhesion molecule 6 (non-specific cross re |
| 151 | XM_045049 | TNFSF10 |
| 152 | XM_007770 | FLJ20171 |
| 153 | NM_015926 | putative secreted protein ZSIG11 |
| 154 | NM_005348 | heat shock 90 kDa protein 1, alpha |
| 155 | NM_003567 | breast cancer anti-estrogen resistance 3 |
| 156 | NM_002507 | nerve growth factor receptor (TNFR superfamily, member 16) |
| 157 | XM_029216 | APEX2 |
| 158 | NM_005654 | nuclear receptor subfamily 2, group F, member 1 |
| 159 | XM_009873 | MMP11 |
| 160 | NM_002105 | H2A histone family, member X |
| 161 | NM_001827 | CDC28 protein kinase regulatory subunit 2 |
| 162 | XM_050486 | NOC4 |
| 163 | XM_015513 | SNRPG |
| 164 | AB037759 | eukaryotic translation initiation factor 2 alpha kinase 4 |
| 165 | NM_000122 | excision repair cross-complementing rodent repair deficiency, complementation gr |
| 166 | NM_006218 | phosphoinositide-3-kinase, catalytic, alpha polypeptide |
| 167 | NM_003127 | spectrin, alpha, non-erythrocytic 1 (alpha-fodrin) |
| 168 | NM_031265 | mucin and cadherin-like |
| 169 | NM_016531 | Kruppel-like factor 3 (basic) |
| 170 | NM_002629 | phosphoglycerate mutase 1 (brain) |
| 171 | NM_003152 | signal transducer and activator of transcription 5A |
| 172 | NM_002037 | FYN oncogene related to SRC, FGR, YES |
| 173 | NM_002607 | platelet-derived growth factor alpha polypeptide |
| 174 | XM_003560 | MAD2L1 |
| 175 | NM_052888 | KIAA0563-related gene |
| 176 | NM_001348 | death-associated protein kinase 3 |
| 177 | NM_003883 | histone deacetylase 3 |
| 178 | NM_001659 | ADP-ribosylation factor 3 |
| 179 | NM_033379 | CDC2 |
| 180 | XM_031718 | EHD4 |
| 181 | NM_014977 | apoptotic chromatin condensation inducer in the nucleus |
| 182 | NM_006570 | Ras-related GTP-binding protein |
| 183 | NM_002466 | v-myb myeloblastosis viral oncogene homolog (avian)-like 2 |
| 184 | NM_001949 | E2F transcription factor 3 |
| 185 | XM_018149 | SELT |
| 186 | NM_013277 | Rac GTPase activating protein 1 |
| 187 | NM_014060 | MCT-1 protein |
| 188 | NM_003684 | MAP kinase-interacting serine/threonine kinase 1 |
| 189 | NM_031966 | cyclin B1 |
| 190 | XM_012601 | MNT |
| 191 | NM_005657 | tumor protein p53 binding protein, 1 |
| 192 | XM_051583 | RAF1 |
| 193 | NM_001255 | CDC20 cell division cycle 20 homolog (S. cerevisiae) |
| 194 | NM_030808 | LIS1-interacting protein NUDEL; endooligopeptidase A |
| 195 | NM_032989 | BCL2-antagonist of cell death |
| 196 | XM_011577 | STK17A |
| 197 | NM_003925 | methyl-CpG binding domain protein 4 |
| 198 | NM_016587 | chromobox homolog 3 (HP1 gamma homolog, Drosophila) |
| 199 | NM_006870 | destrin (actin depolymerizing factor) |
| 200 | XM_008313 | LOC146870 |
| 201 | NM_006812 | amplified in osteosarcoma |
| 202 | NM_003183 | a disintegrin and metalloproteinase domain 17 (tumor necrosis factor, alpha, con |
| 203 | XM_052798 | CDC25C |
| 204 | NM_002626 | phosphofructokinase, liver |
| 205 | NM_033292 | caspase 1, apoptosis-related cysteine protease (interleukin 1, beta, convertase) |
| 206 | XM_006961 | CHD4 |
| 207 | NM_000269 | non-metastatic cells 1, protein (NM23A) expressed in |
| 208 | NM_004873 | BCL2-associated athanogene 5 |
| 209 | NM_001034 | ribonucleotide reductase M2 polypeptide |
| 210 | NM_003070 | SWI/SNF related, matrix associated, actin dependent regulator of chromatin, subf |
| 211 | NM_006595 | apoptosis inhibitor 5 |
| 212 | XM_040402 | CPNE3 |
| 213 | NM_007111 | transcription factor Dp-1 |
| 214 | NM_003597 | TGFB inducible early growth response 2 |
| 215 | NM_002741 | protein kinase C-like 1 |
| 216 | NM_021138 | TNF receptor-associated factor 2 |
| 217 | XM_054954 | CCNF |
| 218 | NM_003879 | CASP8 and FADD-like apoptosis regulator |
| 219 | NM_002089 | chemokine (C-X-C motif) ligand 2 |
| 220 | BC018118 | Rho GTPase activating protein 1 |
| 221 | XM_007070 | TBC1D4 |
| 222 | NM_032094 | protocadherin gamma subfamily A, 12 |
| 223 | NM_003472 | DEK oncogene (DNA binding) |
| 224 | XM_036063 | LOC204666 |
| 225 | XM_006197 | E2IG4 |
| 226 | NM_002198 | interferon regulatory factor 1 |
| 227 | NM_003639 | inhibitor of kappa light polypeptide gene enhancer in B-cells, kinase gamma |
| 228 | XM_010826 | LOC150584 |
| 229 | NM_006393 | nebulette |
| 230 | NM_020436 | sal-like 4 (Drosophila) |
| 231 | XM_038427 | FES |
| 232 | NM_032984 | caspase 2, apoptosis-related cysteine protease (neural precursor cell expressed, |
| 233 | NM_002093 | glycogen synthase kinase 3 beta |
| 234 | XM_043782 | E2F4 |
| 235 | XM_058230 | JUND |
| 236 | XM_071388 | PPFIA2 |
| 237 | XM_056931 | B3GNT1 |
| 238 | NM_002357 | MAX dimerization protein 1 |
| 239 | NM_024320 | hypothetical protein MGC11242 |
| 240 | NM_006763 | BTG family, member 2 |
| 241 | NM_000244 | multiple endocrine neoplasia I |
| 242 | XM_017741 | FSCN1 |
| 243 | W02608 | ESTs, Weakly similar to hypothetical protein FLJ20378 [Homo sapiens] [H. sapiens |
| 244 | XM_044910 | SNRPB |
| 245 | NM_033339 | caspase 7, apoptosis-related cysteine protease |
| 246 | NM_001712 | carcinoembryonic antigen-related cell adhesion molecule 1 (biliary glycoprotein) |
| 247 | NM_031993 | protocadherin gamma subfamily A, 1 |
| 248 | NM_002616 | period homolog 1 (Drosophila) |
| 249 | XM_001357 | MYCBP |
| 250 | NM_031295 | Williams Beuren syndrome chromosome region 21 |
| 251 | NM_001110 | a disintegrin and metalloproteinase domain 10 |
| 252 | NM_004359 | cell division cycle 34 |
| 253 | NM_003667 | G protein-coupled receptor 49 |
| 254 | XM_027651 | TNFRSF10B |
| 255 | NM_012165 | F-box and WD-40 domain protein 3 |
| 256 | XM_009475 | AHCY |
| 257 | XM_035145 | LXN |
| 258 | NM_000365 | TPI1 |
| 259 | NM_003994 | KIT ligand |
| 260 | NM_004341 | carbamoyl-phosphate synthetase 2, aspartate transcarbamylase, and dihydroorotase |
| 261 | XM_039754 | RAB10 |
| 262 | AF346509 | NFAT5 |
| 263 | XM_071453 | YWHAE |
| 264 | NM_006701 | similar to S. pombe dim1+ |
| 265 | NM_024854 | hypothetical protein FLJ22028 |
| 266 | NM_004964 | histone deacetylase 1 |
| 267 | NM_007194 | CHK2 checkpoint homolog (S. pombe) |
| 268 | NM_007168 | ATP-binding cassette, sub-family A (ABC1), member 8 |
| 269 | XM_033064 | ST5 |
| 270 | NM_003841 | tumor necrosis factor receptor superfamily, member 10c, decoy without an intrace |
| 271 | XM_031287 | CXCL3 |
| 272 | NM_003535 | H3FJ |
| 273 | U82467 | tubby homolog (mouse) |
| 274 | XM_017134 | BRCA2 |
| 275 | NM_014784 | Rho guanine nucleotide exchange factor (GEF) 11 |
| 276 | NM_005438 | FOS-like antigen 1 |
| 277 | NM_006107 | acid-inducible phosphoprotein |
| 278 | NM_012323 | v-maf musculoaponeurotic fibrosarcoma oncogene homolog F (avian) |
| 279 | XM_002116 | SFN |
| 280 | NM_006286 | transcription factor Dp-2 (E2F dimerization partner 2) |
| 281 | XM_046643 | NXT1 |
| 282 | AA406526 | Homo sapiens mRNA full length insert cDNA clone EUROIMAGE 2344436. |
| 283 | NM_020637 | fibroblast growth factor 22 |
| 284 | NM_005375 | v-myb myeloblastosis viral oncogene homolog (avian) |
| 285 | NM_012466 | tetraspanin TM4-B |
| 286 | XM_002636 | IGFBP2 |
| 287 | AB037845 | Rho-GTPase activating protein 10 |
| 288 | NM_005983 | S-phase kinase-associated protein 2 (p45) |
| 289 | AF308602 | Notch homolog 1, translocation-associated (Drosophila) |
| 290 | NM_014318 | apoptosis related protein |
| 291 | NM_000207 | insulin |
| 292 | XM_043799 | MPZL1 |
| 293 | XM_010208 | PIM2 |
| 294 | XM_045613 | EHD1 |
| 295 | NM_018948 | Gene 33/Mig-6 |
| 296 | XM_015547 | LATS1 |
| 297 | NM_014248 | ring-box 1 |
| 298 | NM_003558 | phosphatidylinositol-4-phosphate 5-kinase, type I, beta |
| 299 | XM_033878 | TIMP1 |
| 300 | NM_007315 | signal transducer and activator of transcription 1, 91 kDa |
| 301 | NM_000679 | adrenergic, alpha-1B-, receptor |
| 302 | XM_036588 | SDCCAG33 |
| 303 | NM_004078 | cysteine and glycine-rich protein 1 |
| 304 | XM_050512 | ACVR1 |
| 305 | XM_028205 | GLP1R |
| 306 | XM_071498 | E2F6 |
| 307 | AA100736 | hypothetical protein DKFZp434D0215 |
| 308 | NM_005253 | FOS-like antigen 2 |
| 309 | XM_041335 | SCAP2 |
| 310 | AF110908 | TNF receptor-associated factor 3 |
| 311 | XM_058227 | ZK1 |
| 312 | XM_049776 | DSCAM |
| 313 | XM_045802 | PXN |
| 314 | XM_058125 | UBF-fl |
| 315 | NM_005385 | natural killer-tumor recognition sequence |
| 316 | NM_002745 | mitogen-activated protein kinase 1 |
| 317 | XM_031413 | TIAF1 |
| 318 | NM_020249 | a disintegrin-like and metalloprotease (reprolysin type) with thrombospondin typ |
| 319 | XM_046179 | ID1 |
| 320 | XM_007245 | YY1 |
| 321 | AI972873 | SH3 domain binding glutamic acid-rich protein like 2 |
| 322 | XM_047494 | UGDH |
| 323 | NM_022161 | baculoviral IAP repeat-containing 7 (livin) |
| 324 | NM_004493 | hydroxyacyl-Coenzyme A dehydrogenase, type II |
| 325 | XM_009915 | LIF |
| 326 | BF343776 | glutathione reductase |
| 327 | NM_004725 | BUB3 budding uninhibited by benzimidazoles 3 homolog (yeast) |
| 328 | XM_008855 | NR2F6 |
| 329 | NM_018640 | neuronal specific transcription factor DAT1 |
| 330 | XM_013050 | BIRC4 |
| 331 | XM_003222 | CTNNB1 |
| 332 | NM_016316 | REV1-like (yeast) |
| 333 | NM_012098 | angiopoietin-like 2 |
| 334 | XM_058285 | CD24 |
| 335 | NM_004040 | ras homolog gene family, member B |
| 336 | XM_043785 | NOL3 |
| 337 | NM_032471 | protein kinase (cAMP-dependent, catalytic) inhibitor beta |
| 338 | NM_022873 | interferon, alpha-inducible protein (clone IFI-6-16) |
| 339 | XM_035114 | KIAA1277 |
| 340 | XM_007722 | CHD2 |
| 341 | NM_006054 | reticulon 3 |
| 342 | XM_054920 | KIAA0828 |
| 343 | NM_001895 | casein kinase 2, alpha 1 polypeptide |
| 344 | NM_032365 | PRO2000 |
| 345 | XM_010040 | ARHGAP8 |
| 346 | NM_005419 | signal transducer and activator of transcription 2, 113 kDa |
| 347 | NM_003299 | tumor rejection antigen (gp96) 1 |
| 348 | XM_042423 | EMP1 |
| 349 | AF207547 | LATS, large tumor suppressor, homolog 2 (Drosophila) |
| 350 | NM_002878 | RAD51-like 3 (S. cerevisiae) |
| 351 | XM_010914 | PCAF |
| 352 | XM_038418 | PRC1 |
| 353 | Z18817 | heat shock 70 kDa protein 4 |
| 354 | U70451 | myeloid differentiation primary response gene (88) |
| 355 | NM_002957 | retinoid X receptor, alpha |
| 356 | XM_046041 | CCT2 |
| 357 | XM_028620 | HOXC9 |
| 358 | XM_012894 | ZNF14 |
| 359 | NM_021979 | heat shock 70 kDa protein 2 |
| 360 | NM_005163 | v-akt murine thymoma viral oncogene homolog 1 |
| 361 | XM_006299 | API5 |
| 362 | NM_001388 | developmentally regulated GTP binding protein 2 |
| 363 | NM_004992 | methyl CpG binding protein 2 (Rett syndrome) |
| 364 | XM_016845 | HHGP |
| 365 | AK054731 | tubulin, alpha 1 (testis specific) |
| 366 | XM_003628 | CCNG2 |
| 367 | NM_000291 | phosphoglycerate kinase 1 |
| 368 | XM_044653 | EGFR |
| 369 | XM_046245 | PIG8 |
| 370 | NM_007229 | protein kinase C and casein kinase substrate in neurons 2 |
| 371 | NM_033637 | beta-transducin repeat containing |
| 372 | XM_033862 | ELK1 |
| 373 | NM_000638 | vitronectin (serum spreading factor, somatomedin B, complement S-protein) |
| 374 | NM_018098 | epithelial cell transforming sequence 2 oncogene |
| 375 | NM_001880 | activating transcription factor 2 |
| 376 | NM_003122 | serine protease inhibitor, Kazal type 1 |
| 377 | XM_008055 | COX4I1 |
| 378 | XM_046881 | SLC9A1 |
| 379 | NM_003860 | barrier to autointegration factor 1 |
| 380 | XM_003029 | ITGB5 |
| 381 | NM_005566 | lactate dehydrogenase A |
| 382 | NM_019113 | fibroblast growth factor 21 |
| 383 | XM_030478 | SVIL |
| 384 | NM_006167 | NK3 transcription factor related, locus 1 (Drosophila) |
| 385 | NM_007324 | MAD, mothers against decapentaplegic homolog (Drosophila) interacting protein, r |
| 386 | NM_002342 | lymphotoxin beta receptor (TNFR superfamily, member 3) |
| 387 | NM_002909 | regenerating islet-derived 1 alpha (pancreatic stone protein, pancreatic thread |
| 388 | XM_041552 | RAD17 |
| 389 | NM_030662 | mitogen-activated protein kinase kinase 2 |
| 390 | NM_022333 | TIA1 cytotoxic granule-associated RNA binding protein-like 1 |
| 391 | XM_037682 | SMARCB1 |
| 392 | XM_033932 | FLJ20485 |
| 393 | BC002513 | eukaryotic translation initiation factor 2, subunit 1 alpha, 35 kDa |
| 394 | NM_003470 | ubiquitin specific protease 7 (herpes virus-associated) |
| 395 | NM_001320 | casein kinase 2, beta polypeptide |
| 396 | AA527919 | Homo sapiens, clone IMAGE: 5285034, mRNA |
| 397 | NM_005167 | hypothetical protein MGC19531 |
| 398 | XM_045642 | SF1 |
| 399 | XM_029816 | YWHAB |
| 400 | NM_006121 | keratin 1 (epidermolytic hyperkeratosis) |
| 401 | NM_004843 | class I cytokine receptor |
| 402 | NM_000450 | selectin E (endothelial adhesion molecule 1) |
| 403 | NM_013374 | programmed cell death 6 interacting protein |
| 404 | AK024858 | hypothetical protein LOC221496 |
| 405 | XM_006890 | ELK3 |
| 406 | NM_022870 | myosin, heavy polypeptide 11, smooth muscle |
| 407 | XM_033910 | TCP1 |
| 408 | XM_030523 | MAP3K8 |
| 409 | NM_003821 | receptor-interacting serine-threonine kinase 2 |
| 410 | XM_002633 | MYCN |
| 411 | NM_002087 | granulin |
| 412 | NM_007019 | ubiquitin-conjugating enzyme E2C |
| 413 | AI685200 | DKFZP586G1517 protein |
| 414 | XM_009203 | AKT2 |
| 415 | NM_013986 | Ewing sarcoma breakpoint region 1 |
| 416 | NM_004208 | programmed cell death 8 (apoptosis-inducing factor) |
| 417 | XM_011791 | LAMC3 |
| 418 | NM_022746 | hypothetical protein FLJ22390 |
| 419 | AL042759 | NADPH oxidase organizer 1 |
| 420 | NM_003808 | tumor necrosis factor (ligand) superfamily, member 13 |
| 421 | XM_002562 | VAMP5 |
| 422 | NM_005923 | mitogen-activated protein kinase kinase kinase 5 |
| 423 | NM_001315 | mitogen-activated protein kinase 14 |
| 424 | NM_007022 | putative tumor suppressor 101F6 |
| 425 | XM_047007 | PLAGL2 |
| 426 | NM_005556 | keratin 7 |
| 427 | NM_000454 | superoxide dismutase 1, soluble (amyotrophic lateral sclerosis 1 (adult)) |
| 428 | AI886326 | hypothetical protein FLJ21195 similar to protein related to DAC and cerberus |
| 429 | NM_005917 | malate dehydrogenase 1, NAD (soluble) |
| 430 | NM_002835 | protein tyrosine phosphatase, non-receptor type 12 |
| 431 | NM_005972 | pancreatic polypeptide receptor 1 |
| 432 | NM_016328 | GTF2I repeat domain containing 1 |
| 433 | NM_000860 | hydroxyprostaglandin dehydrogenase 15-(NAD) |
| 434 | NM_003882 | WNT1 inducible signaling pathway protein 1 |
| 435 | XM_028817 | ADCY6 |
| 436 | NM_000955 | prostaglandin E receptor 1 (subtype EP1), 42 kDa |
| 437 | X68560 | Sp3 transcription factor |
| 438 | NM_006443 | putative c-Myc-responsive |
| 439 | NM_001090 | ATP-binding cassette, sub-family F (GCN20), member 1 |
| 440 | NM_002827 | protein tyrosine phosphatase, non-receptor type 1 |
| 441 | XM_034007 | BCAR1 |
| 442 | NM_005901 | MAD, mothers against decapentaplegic homolog 2 (Drosophila) |
| 443 | NM_001963 | epidermal growth factor (beta-urogastrone) |
| 444 | BM044930 | neuronal guanine nucleotide exchange factor |
| 445 | NM_004701 | cyclin B2 |
| 446 | XM_002375 | IL1F8 |
| 447 | NM_001945 | diphtheria toxin receptor (heparin-binding epidermal growth factor-like growth f |
| 448 | NM_000230 | leptin (obesity homolog, mouse) |
| 449 | NM_001903 | catenin (cadherin-associated protein), alpha 1, 102 kDa |
| 450 | NM_002220 | inositol 1,4,5-trisphosphate 3-kinase A |
| 451 | NM_020384 | claudin 2 |
| 452 | NM_002734 | protein kinase, cAMP-dependent, regulatory, type I, alpha (tissue specific extin |
| 453 | NM_020243 | translocase of outer mitochondrial membrane 22 homolog (yeast) |
| 454 | NM_004380 | CREB binding protein (Rubinstein-Taybi syndrome) |
| 455 | XM_044659 | CSK |
| 456 | NM_002875 | RAD51 homolog (RecA homolog, E. coli) (S. cerevisiae) |
| 457 | XM_033428 | AK1 |
| 458 | NM_005745 | accessory protein BAP31 |
| 459 | NM_030753 | wingless-type MMTV integration site family, member 3 |
| 460 | XM_034587 | FLJ22174 |
| 461 | NM_004920 | AATK |
| 462 | NM_007065 | CDC37 cell division cycle 37 homolog (S. cerevisiae) |
| 463 | NM_001239 | cyclin H |
| 464 | XM_036323 | TSG101 |
| 465 | NM_001233 | caveolin 2 |
| 466 | XM_015956 | CTBP2 |
| 467 | XM_015505 | AXL |
| 468 | NM_003749 | insulin receptor substrate 2 |
| 469 | XM_016033 | DPF3 |
| 470 | NM_004889 | ATP synthase, H+ transporting, mitochondrial F0 complex, subunit f, isoform 2 |
| 471 | XM_003213 | NS |
| 472 | XM_033761 | COBL |
| 473 | XM_047049 | E2F1 |
| 474 | NM_006572 | guanine nucleotide binding protein (G protein), alpha 13 |
| 475 | NM_006024 | Tax1 (human T-cell leukemia virus type I) binding protein 1 |
| 476 | NM_016245 | retinal short-chain dehydrogenase/reductase 2 |
| 477 | XM_010339 | GPC4 |
| 478 | NM_002129 | high-mobility group box 2 |
| 479 | NM_006565 | CCCTC-binding factor (zinc finger protein) |
| 480 | AL137667 | MAPK8 |
| 481 | XM_050236 | LENG4 |
| 482 | NM_005805 | 26S proteasome-associated pad1 homolog |
| 483 | XM_054928 | CLN8 |
| 484 | NM_001350 | death-associated protein 6 |
| 485 | NM_016073 | likely ortholog of mouse hepatoma-derived growth factor, related protein 3 |
| 486 | XM_031926 | NFKB2 |
| 487 | NM_005085 | nucleoporin 214 kDa |
| 488 | NM_003904 | zinc finger protein 259 |
| 489 | NM_014397 | NIMA (never in mitosis gene a)-related kinase 6 |
| 490 | XM_017096 | ABR |
| 491 | XM_003477 | FAT |
| 492 | NM_001982 | v-erb-b2 erythroblastic leukemia viral oncogene homolog 3 (avian) |
| 493 | NM_006705 | growth arrest and DNA-damage-inducible, gamma |
| 494 | NM_004958 | FK506 binding protein 12-rapamycin associated protein 1 |
| 495 | XM_004713 | FLNC |
| 496 | NM_021235 | epidermal growth factor receptor substrate EPS15R |
| 497 | XM_030044 | CSE1L |
| 498 | AI685466 | LOC90353 |
| 499 | NM_003311 | tumor suppressing subtransferable candidate 3 |
| 500 | XM_039984 | CNOT8 |
| 501 | XM_001831 | CYR61 |
| 502 | XM_052827 | CFL2 |
| 503 | XM_007487 | ASB2 |
| 504 | XM_003405 | HD |
| 505 | XM_012723 | C18orf1 |
| 506 | NM_005564 | lipocalin 2 (oncogene 24p3) |
| 507 | XM_010767 | NCKAP1 |
| 508 | NM_001324 | cleavage stimulation factor, 3′ pre-RNA, subunit 1, 50 kDa |
| 509 | NM_005658 | TNF receptor-associated factor 1 |
| 510 | NM_000168 | GLI-Kruppel family member GLI3 (Greig cephalopolysyndactyly syndrome) |
| 511 | XM_027639 | DKFZP434J214 |
| 512 | XM_033445 | SLC7A7 |
| 513 | NM_000852 | glutathione S-transferase pi |
| 514 | NM_002097 | general transcription factor IIIA |
| 515 | NM_003243 | transforming growth factor, beta receptor III (betaglycan, 300 kDa) |
| 516 | XM_003444 | FGF5 |
| 517 | XM_035107 | BRAF |
| 518 | D55886 | adenylate cyclase 5 |
| 519 | NM_005633 | son of sevenless homolog 1 (Drosophila) |
| 520 | AI161049 | voltage-dependent calcium channel gamma subunit-like protein |
| 521 | XM_045460 | CDC25B |
| 522 | AA634799 | Homo sapiens cDNA: FLJ22864 fis, clone KAT02164. |
| 523 | NM_004230 | endothelial differentiation, sphingolipid G-protein-coupled receptor, 5 |
| 524 | XM_040912 | AMN |
| 525 | XM_056595 | OTOF |
| 526 | XM_054160 | VMD2 |
| 527 | XM_049935 | CTEN |
| 528 | NM_006365 | transcriptional activator of the c-fos promoter |
| 529 | XM_027186 | WNT2 |
| 530 | NM_001067 | topoisomerase (DNA) II alpha 170 kDa |
| 531 | XM_044785 | KCNJ13 |
| 532 | XM_007585 | TJP1 |
| 533 | XM_042940 | UNC5C |
| 534 | XM_037408 | BAP1 |
| 535 | XM_005428 | 1-Dec |
| 536 | NM_014452 | tumor necrosis factor receptor superfamily, member 21 |
| 537 | NM_006645 | serologically defined colon cancer antigen 28 |
| 538 | XM_031972 | CNNM2 |
| 539 | XM_047561 | ARHA |
| 540 | XM_046191 | CGI-31 |
| 541 | NM_003778 | UDP-Gal: betaGlcNAc beta 1,4-galactosyltransferase, polypeptide 4 |
| 542 | XM_011713 | COPS5 |
| 543 | NM_032957 | tumor necrosis factor receptor superfamily, member 6b, decoy |
| 544 | NM_006044 | histone deacetylase 6 |
| 545 | NM_021144 | PC4 and SFRS1 interacting protein 1 |
| 546 | AA531287 | ESTs |
| 547 | XM_033355 | ABL1 |
| 548 | XM_008394 | EZH1 |
| 549 | XM_036570 | TNFRSF12A |
| 550 | XM_031209 | IL1F9 |
| 551 | XM_027311 | BFAR |
| 552 | NM_006166 | nuclear transcription factor Y, beta |
| 553 | XM_043103 | HSD11B2 |
| 554 | XM_050735 | ST14 |
| 555 | NM_057159 | endothelial differentiation, lysophosphatidic acid G-protein-coupled receptor, 2 |
| 556 | NM_001702 | brain-specific angiogenesis inhibitor 1 |
| 557 | NM_005312 | guanine nucleotide-releasing factor 2 (specific for crk proto-oncogene) |
| 558 | NM_001042 | solute carrier family 2 (facilitated glucose transporter), member 4 |
| 559 | L41944 | interferon (alpha, beta and omega) receptor 2 |
| 560 | NM_000264 | patched homolog (Drosophila) |
| 561 | XM_041744 | IER3 |
| 562 | NM_005967 | NGFI-A binding protein 2 (EGR1 binding protein 2) |
| 563 | XM_009170 | CEACAM7 |
| 564 | NM_004231 | ATPase, H+ transporting, lysosomal 14 kDa, V1 subunit F |
| 565 | NM_004315 | N-acylsphingosine amidohydrolase (acid ceramidase) 1 |
| 566 | XM_008654 | MAP2K4 |
| 567 | XM_041847 | TNF |
| 568 | XM_040448 | RAD1 |
| 569 | XM_011068 | MST1R |
| 570 | NM_000662 | N-acetyltransferase 1 (arylamine N-acetyltransferase) |
| 571 | XM_001744 | TNFRSF8 |
| 572 | XM_028038 | BMPR2 |
| 573 | NM_006534 | nuclear receptor coactivator 3 |
| 574 | NM_005091 | peptidoglycan recognition protein |
| 575 | NM_024426 | Wilms tumor 1 |
| 576 | AA290601 | hypothetical protein LOC137075 |
| 577 | AI810669 | ESTs, Moderately similar to hypothetical protein FLJ20378 [Homo sapiens] [H. sap |
| 578 | NM_003550 | MAD1 mitotic arrest deficient-like 1 (yeast) |
| 579 | NM_012415 | RAD54B homolog |
| 580 | XM_033469 | TGFBR2 |
| 581 | XM_039779 | CAPRI |
| 582 | XM_049512 | TRIP13 |
| 583 | NM_002969 | mitogen-activated protein kinase 12 |
| 584 | NM_005380 | neuroblastoma, suppression of tumorigenicity 1 |
| 585 | XM_029490 | DPH2L1 |
| 586 | AL136835 | Toll-interacting protein |
| 587 | XM_034567 | CCND2 |
| 588 | NM_032192 | protein phosphatase 1, regulatory (inhibitor) subunit 1B (dopamine and cAMP regu |
| 589 | NM_000072 | CD36 antigen (collagen type I receptor, thrombospondin receptor) |

1. A compound having the structure of Formula (I) 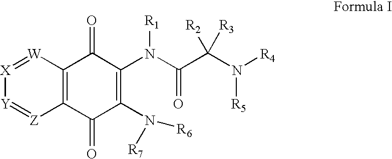
wherein
W, X, Y and Z are each selected from a bond, CH, C—R₈, C—R₉, C—R₁₀, C—R₁₁, O (oxygen), N (nitrogen) and S (sulfur) and no more than two of W, X, Y and Z are simultaneously O, N and S;
wherein, R₈, R₉, R₁₀, R₁₁ are each selected from hydrogen, hydroxyl, sulfhydryl, alkoxy, thioalkoxy, alkyl, halogen, CN, CF₃, NO₂, COOR₁₂, CONR₁₂R₁₃, NR₁₂R₁₃, NR₁₂COR₁₃, NR₁₂SO₂R₁₃ and NR₁₄CONR₁₂R₁₃;
wherein R₁₂, R₁₃ and R₁₄ are each selected from hydrogen, alkyl, heteroalkyl, aryl, arylalkyl, heteroaryl, heteroarylalkyl, cycloalkyl and heterocycloalkyl;
and wherein NR₁₂R₁₃ is further optionally selected from substituted and unsubstituted mono or bicyclic ring with one to four heteroatoms such as N, O and S;
and further wherein R₁₂ and R₁₄ may form a 4, 5, 6 or 7-membered cyclic ring system;
wherein R₁, R₂, R₃, R₄, and R₅ are each selected from hydrogen, alkyl, substituted or unsubstituted phenyl, substituted or unsubstituted polyaromatic ring, substituted or unsubstltuted heteroaromatic ring having hetero atom(s) selected from N, O and S, substituted or unsubstituted aralkyl, substituted or unsubstituted, cyclic or polycyclic, hydrocarbon and substituted or unsubstituted, monoheterocycle or polyheterocycle (of 3-8 atoms per ring) having one to four hetero atoms selected from N, O, and S; and
and wherein said substitutions are selected from hydrogen, hydroxyl, sulfhydryl, alkoxy, thioalkoxy, alkyl, halogen, CN, CF₃, NO₂, COOR₁₂, CONR₁₂R₁₃, NR₁₂R₁₃, NR₁₂COR₁₃, NR₁₂SO₂R₁₃, and NR₁₄CONR₁₂R₁₃;
wherein R₁₂, R₁₃ and R₁₄ are each selected from hydrogen, alkyl, heteroalkyl, aryl, arylalkyl, heteroaryl, heteroarylalkyl, cycloalkyl, and heterocycloalkyl;
and wherein NR₁₂R₁₃ may form a substituted and unsubstituted, mono or bicyclic ring with one to four heteroatoms selected from N, O and S;
and wherein R₁₂ and R₁₄ may 1053 form a 4, 5, 6 or 7-member cyclic ring system;
and wherein R₁, R₄, R₅, R₆ and R₇ are also selected from: 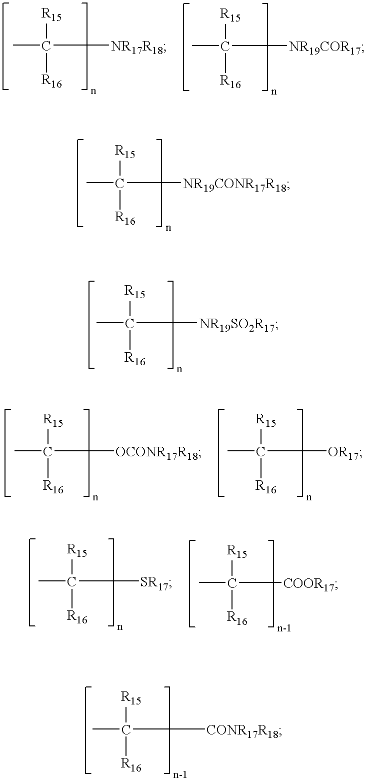
where n is 2, 3 or 4 and R₁₅, R₁₆, R₁₇, R₁₈ and R₁₉ are each selected from hydrogen, alkyl, cycloalkyl, unsubstituted or substituted aryl, unsubstituted or substituted heteroaryl, and unsubstituted or substituted alkylaryl;
NR₁₇R₁₈ may form a substituted or unsubstituted, mono or bicyclic ring with one to four heteroatoms selected from N, O and S; and wherein R₁₇ and R₁₉ may form a 4, 5, 6 or 7-membered cyclic ring system;
and wherein R₄ may also be selected from —COR₁₇, —SO₂R₁₇, —CONR₁₇R₁₈ and —C(═NR₁₉)NR₁₇R₁₈;
and wherein R₆ and R₇ are each selected from:
alkyl, substituted and unsubstituted phenyl or polyaromatic, substituted or unsubstituted heteroaromatic, wherein said hetero atom is selected from N, O and S, substituted or unsubstituted aralkyl, and substituted or unsubstituted, cyclic or polycyclic hydrocarbon, or mono-or poly-heterocycle of 3 to 8 atom rings having one to four hetero atoms selected from N, O and S; and
wherein said substitutions are selected from hydroxyl, sulfhydryl, alkoxy, thioalkoxy, alkyl, halogen, CN, CF₃, NO₂, COOR₁₂, CONR₁₂R₁₃, NR₁₂R₁₃, NR₁₂COR₁₃, NR₁₂SO₂R₁₃ and NR₁₄CONR₁₂R₁₃;
wherein R₁₂, R₁₃ and R₁₄ are each selected from hydrogen, alkyl, heteroalkyl, aryl, arylalkyl, heteroaryl, heteroarylalkyl, cycloalkyl, and heterocycloalkyl;
and wherein NR₁₂R₁₃ may form a substituted or unsubstituted, mono or bicyclic, ring with one to four heteroatoms selected from N, O and S;
and wherein NR₄R₅ and NR₆R₇ may each be selected from substituted and unsubstituted, mono or bicyclic, rings comprising one to four heteroatoms selected from N, O and S and wherein said N may also be substituted or unsubstituted,
and including salts thereof.

2. The compound of claim 1 wherein W and Z are each C—R₈, C—R₁₁ or N and wherein X and Y are each C—R₉ or C—R₁₀.

3. The compound of claim 1 wherein X and Y are each C—R₉, C—R₁₀ or N and wherein W and Z are each C—R₈ or C—R₁₁.

4. The compound of claim 1 wherein W is C—R₈ or N and wherein X, Y and Z are each C—R₉, C—R₁₀ or C—R₁₁.

5. The compound of claim 1 wherein Z is C—R₁₁ or N and wherein W, Y and Z are each C—R₈, C—R₉ or C—R₁₀.

6. The compound of claim 1 wherein X is C—R₉ or N and wherein W, Y and Z are each C—R₈, C—R₁₀ or C—R₁₁.

7. The compound of claim 1 wherein Y is C—R₁₀ or N and wherein W, X, and Z are each CH, C—R₈, C—R₉ or C—R₁₁.

8. The compound of claim 1 wherein W, X, Y and Z are each selected from CH, C—R₈, C—R₉, C—R₁₀ and C—R₁₁.

9. The compound of claim 8 wherein W, X, Y and Z are each CH.

10. The compound of claim 1 wherein R₂ and R₃ are each selected from hydrogen, lower alkyl of 1-6 carbons and aryl.

11. The compound of claim 1 wherein R₁ is selected from hydrogen, alkyl, cycloalkyl, unsubstituted or substituted phenyl, unsubstituted or substituted benzyl, -methylpyridine, -ethylpyridine, -methylindole, -ethylindole, alkoxyethyl-, hydroxyethyl-, N,N-dialkyl-ethyl, N,N-dialkyl-propyl, methylpyrrole, -ethylpyrrole, -methylfuran, -ethylfuran, -alkylmorpholine, -alkylpiperizine, -alkypiperidine, and -alkylpyrrolidine, and wherein R₂ and R₃ are each hydrogen, lower alkyl (1-6 carbon) or aryl.

12. The compound of claim 1 wherein R₄ and R₅ are each selected from hydrogen, alkyl, cycloalkyl, unsubstituted or substituted phenyl, unsubstituted or substituted benzyl, -methylpyridine, -ethylpyridine, -methylindole, -ethylindole, alkoxyethyl-, hydroxyethyl-, N,N-dialkyl-ehthl, N,N-dialkyl-propyl, -methylpyrrole, -ethylpyrrole, -methylfuran, -ethylfuran, --alkylmorpholine, -alkylpiperizine, -alkypiperidine, and -alkylpyrrolidine, or wherein —NR₄R₅ is a substituted or unsubstituted, monocyclic or bicyclic, heterocycloalkyl ring, and wherein R₂ and R₃ are each selected from hydrogen, lower alkyl (1-6 carbon) and aryl.

13. The compound of claim 1 wherein R₆ and R₇ are selected from alkyl, cycloalkyl, unsubstituted or substituted phenyl, unsubstituted or substituted benzyl, -methylpyridine, -ethylpyridine, -methylindole, -ethylindole, alkoxyethyl-, hydroxyethyl-, N,N-dialkyl-ethyl, N,N-dialkyl-propyl, -methylpyrrole, -ethylpyrrole, -methylfuran, -ethylfuran, -alkylmorpholine, -alkylpiperizine, -alkypiperidine, and -alkylpyrrolidine, and R₂ and R₃ are selected from hydrogen, lower alkyl (1-6 carbon) and aryl.

14. The compound of claim 9 wherein R₂ and R₃ are selected from hydrogen, lower alkyl (1-6 carbon) and aryl.

15. The compound of claim 9 wherein R₁, R₄ and R₅ are each selected from hydrogen, alkyl, cycloalkyl, unsubstituted or substituted phenyl, unsubstituted or substituted benzyl, methylpyridine, -ethylpyridine, -methylindole, -ethylindole, alkoxyethyl-, hydroxyethyl-, N,N-dialkyl-ethyl, N,N-dialkyl-propyl, -methylpyrrole, -ethylpyrrole, -methylfuran, -ethylfuran, -alkylmorpholine, -alkylpiperizine, -alkypiperidine, and -alkylpyrrolidine, or wherein —NR₄R₅ is a substituted or unsubstituted, monocyclic or bicyclic, heterocycloalkyl ring, and wherein R₂ and R₃ are each hydrogen, lower alkyl (1-6 carbon) or aryl and wherein R₆ and R₇ are each selected from alkyl, cycloalkyl, unsubstituted or substituted phenyl, unsubstituted or substituted benzyl, -methylpyridine, -ethylpyridine, -methylindole, -ethylindole, alkoxyethyl-, hydroxyethyl, N,N-dialkyl-ethyl, N,N-dialkyl-propyl, -methylpyrrole, -ethylpyrrole, -methylfuran, -ethylfuran, -alkymorpholine, -alkylpiperizine, -alkypiperidine, and -alkylpyrrolidine.

16. The compound of claim 1 wherein R₂ and R₃ are each selected from hydrogen and alkyl, and wherein R₄ and R₆ are each selected from alkyl and 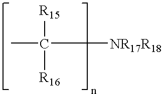
where n is 2 ,3 or 4 and one or both of R₅ and R₇ is alkyl.

17. The compound of claim 9 wherein R₁ is alkyl, wherein R₂ and R₃ are each selected from hydrogen and alkyl and wherein R₄ and R₆ are each selected from alkyl and 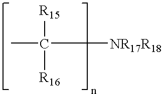
wherein n is 2, 3 or 4 and one or both of R₅ and R₇ is alkyl.

18. The compound of claim 1 wherein R₂ and R₃ are each selected from hydrogen and alkyl, wherein R₄ and R₆ are each selected from alkyl and 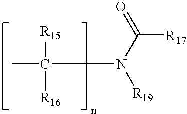
wherein n is 2, 3 or 4 and one or both of R₅ and R₇ is alkyl.

19. The compound of claim 1 wherein
R₂ and R₃ are each selected from hydrogen and alkyl
wherein R₄ and R₆ are each selected from alkyl and 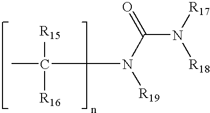
where n is 2, 3 or 4 and one or both of R₅ and R₇ is alkyl

20. Compound of claim 1
wherein
R₂ and R₃ are each selected from hydrogen and alkyl
R₄ and R₆ are each selected from alkyl and 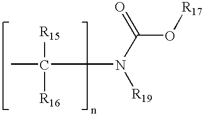
where n is 2, 3 or 4 and one or both of R₅ and R₇ is alkyl.

21. The compound of claim 1 wherein
R₂ and R₃ are each selected from hydrogen and alkyl
R₄ and R₆ are each selected from alkyl and 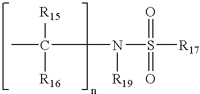
where n is 2, 3 or 4 and one or both of R₅ and R₇ is alkyl.

22. The compound of claim 1 wherein
R₂ and R₃ are each selected from hydrogen and alkyl,
R₄ and R₆ are each selected from alkyl and 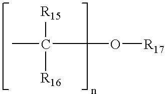
where n is 2, 3 or 4 and one or both of R₅ and R₇ is alkyl.

23. The compound of claim 1 wherein R₁ is methyl.

24. The compound of claim 9 wherein R₁ is methyl.

25. The compound of claim 1 wherein one or more of R₁, R₆ and R₇ is methyl.

26. The compound of claim 9 wherein one or more of R₁, R₆, and R₇ is methyl.

27. The compound of claim 23 wherein —NR₄R₅ is a substituted or unsubstituted, monocyclic or bicyclic, heterocycloalkyl ring.

28. The compound of claim 25 wherein —NR₄R₅ is a substituted or unsubstituted, monocyclic or bicyclic, heterocycloalkyl ring.

29. The compound of claim 26 wherein —NR₄R₅ is a substituted or unsubstituted, monocyclic or bicyclic, heterocycloalkyl ring.

30. The compound of claim 26 wherein —NR₄R₅ is selected from aziridine, pyrrolidine, piperidine, hydroxy piperidine, morpholine, and N-methyl piperazine.

31. The compound of claim 23 wherein R₄ and R₅ are each lower alkylene-OR₂₀ wherein R₂₀ is hydrogen or lower alkyl.

32. The compound of claim 25 wherein R₄ and R₅ are each lower alkylene-OR20 wherein R₂₀ is hydrogen or lower alkyl.

33. The compound of claim 26 wherein R₄ and R₅ are each lower alkylene-OR₂₀ wherein R₂₀ is hydrogen or lower alkyl.

34. A compound of claim 1 having a structure of Table 1 including salts thereof.

35. A compound of claim 1 having a structure of Table 2 including salts thereof.

36. A compound of claim 1 having a structure of Table 3 including salts thereof.

37. A compound of claim 1 having a structure of Table 4 including salts thereof.

38. A compound of claim 1 having a structure of Table 5 including salts thereof.

39. A compound of claim 1 having a structure of Table 6 including salts thereof.

40. A compound having a structure of Table 7 or Table 8 including salts thereof.

41. A compound having a structure of Table 9 or Table 10 including salts thereof.

42. A compound having a structure of Table 11 or Table 12 including salts thereof.

43. A pharmaceutical composition comprising a therapeutically effective amount of a compound of claim 1 in a pharmaceutically acceptable carrier.

44. A method for preventing or treating a disease associated with a change in levels of expression of a set of genes in a mammal comprising administering to said mammal an effective amount of a compound of claim 1.

45. A method for preventing or treating a disorder modulated by altered gene expression, wherein the disorder is selected from the group consisting of cancer, cardiovascular disorders, arthritis, osteoporosis, inflammation, periodontal disease and skin disorders, by administering to a mammal in need of such treatment a safe and effective amount of a compound according to claim 1.

46. The method of claim 45, wherein the disorder is cancer.

47. The method of claim 46 wherein said treatment prevents, arrests or reverts tumor growth and metastasis.

48. The method of claim 46 wherein said cancer is selected from the group consisting of solid tumors, lymphomas, skin cancer, urinary bladder cancer, breast cancer, uterine cancer, ovarian cancer, prostate cancer, lung cancer, colon cancer, rectum cancer, pancreatic cancer, kidney cancer, and stomach cancer.

49. The method of claim 48 wherein the cancer is breast or colon cancer.

50. The method of claim 49 wherein said breast or colon cancer is adenocarcinoma.

51. The method of claim 45 wherein the disorder is a cardiovascular disorder selected from the group consisting of dilated cardiomyopathy, congestive heart failure, atherosclerosis, plaque rupture, reperfusion injury, ischemia, chronic obstructive pulmonary disease, angioplasty restenosis, and aortic aneurysm.

52. A gene set wherein expression of each member of said gene set is modulated as a result of treatment with a compound of claim 1.

53. The gene set of claim 52 wherein expression of each member of said gene set is increased or each member of said gene set is decreased as a result of said treatment.

54. The gene set of claim 52 wherein the members of said gene set are selected from the genes identified in Table 19.

55. The gene set of claim 52 wherein said gene set is present in a cell.

56. A method for identifying an agent that modulates the expression of a gene set of claim 51, comprising:
(a) contacting a compound with a test system containing one or more polynucleotides corresponding to each of the members of the gene set of claim 52 under conditions wherein the members of said gene set are being expressed; (b) determining a change in expression of each of said one or more polynucleotides of step (a) as a result of said contacting;
wherein said change in expression in step (b) indicates modulation of the members of said gene set thereby identifying said test compound as an agent that modulates the expression of said gene set.

57. The method of claim 56 wherein said change in expression is a decrease in expression of said one or more polynucleotides.

58. The method of claim 56 wherein said change in expression is a change in transcription of said one or more polynucleotides.

59. The method of claim 56 wherein said change in expression is determined by determining a change in activity of a polypeptide encoded by said polynucleotide.

60. The method of claim 56 wherein said one or more polynucleotides are present in a cell.

61. The method of claim 60 wherein said cell is a cancer cell.

62. The method of claim 60 wherein said cancer cell is a breast or colon cancer cell.

63. The method of claim 62 wherein said breast or colon cancer cell is an adenocarcinoma cancer cell.

64. The method of claim 60 wherein said cell is a recombinant cell engineered to contain said set of genes.

65. A set of genes comprising a plurality of subsets of genes wherein each subset of said plurality is a gene set identified by the method of claim 56.

66. Compounds identified as having activity using the method of claim 56.

67. The gene set of claim 51 wherein said gene set comprises a subset of the genes of Table 19.

68. The method of claim 56 wherein said compound modulates the expression of a subset of genes of Table 19.

69. A compound of claim 1 and having a structure of Table 13 and Table 14 including salts thereof.

70. A compound of claim 1 and having a structure of Table 15 and Table 16 including salts thereof.

71. A compound of claim 1 and having a structure of Table 17 and Table 18 including salts thereof.
